(19) United States
(12) Patent Application Publication
(10) Pub. No.: US 2026/0248761 A1
(43) Pub. Date: Aug. 27, 2026

(54) COMPOSITIONS AND METHODS FOR TREATMENT OF PULMONARY HYPERTENSION

(71) Applicant: EpicentRx, Inc., La Jolla, CA (US)

(72) Inventors: Bryan ORONSKY, Los Altos Hills, CA (US); Scott CAROEN, San Francisco, CA (US); Tony R. REID, San Diego, CA (US)

(73) Assignee: EpicentRx, Inc., La Jolla, CA (US)

(21) Appl. No.: 18/861,730

(22) PCT Filed: May 1, 2023
(86) PCT No.: PCT/US2023/020574

§ 371 Date: Oct. 30, 2024

Related U.S. Application Data
(60) Provisional application No. 63/337,540, filed on May 2, 2022.
(60) Provisional application No. 63/422,823, filed on Nov. 4, 2022.

(51) Int. Cl. A61K 31/397

(52) U.S. Cl. CPC A61K 31/397

(57) ABSTRACT

A method of treating pulmonary hypertension (PH) and the consequences of PH on the right side of the heart in a patient is provided. The method includes administering a therapeutically effective dose of RRx-001, or a pharmaceutically acceptable salt thereof, either alone or in combination with a blood product and/or an agent, such as an agent for treating PH.

### CROSS-REFERENCE TO RELATED APPLICATIONS

This application claims the benefit of and priority to U.S. Provisional Patent Application Ser. No. 63/337,540 filed on May 2, 2022 and U.S. Provisional Patent Application Ser. No. 63/422,823 filed on Nov. 4, 2022, the contents of which are hereby incorporated by reference in their entirety.

### FIELD OF THE INVENTION

This disclosure relates to compositions and methods of treatment for pulmonary hypertension (PH), such as pulmonary arterial hypertension (PAH). The methods include administering a therapeutically effective amount of RRx-001, or a pharmaceutically acceptable salt thereof, to a subject in need thereof, individually or in combination with a blood product and/or an agent, resulting in an improvement in at least one symptom associated with the PH or the PAH in the subject.

### BACKGROUND

PH is the term used to describe a group of disorders characterized by abnormally high pressures in the pulmonary arteries. PAH is a subtype of PH and is a serious, life-threatening, life-shortening, and progressive medical condition, most often encountered in patients with left heart failure or severe chronic obstructive pulmonary disease (COPD). It occurs in an idiopathic or primary form or secondarily in association with other disease states or exposures and is defined as an elevated pulmonary artery pressure >25 mmHg, which inexorably leads to right ventricular dysfunction and failure, if left untreated. See “Managing pulmonary hypertension and cor pulmonale: Chronic obstructive pulmonary disease in over 16s: diagnosis and management: Evidence review A. London: National Institute for Health and Care Excellence (NICE); 2018 December (NICE Guideline, No. 115.) Available from: https://www.ncbi.nlm.nih.gov/books/NBK560180/. The major determinant of morbidity and mortality in P H is right ventricular dysfunction and failure (RVF). See D'Alonzo G E, Barst R J, Ayres S M, Bergofsky E H, Brundage B H, Detre K M, Fishman A P, Goldring R M, Groves B M, Kernis J T, et al., Survival in patients with primary pulmonary hypertension: results from a national prospective registry. Ann Intern Med 1991; 115:343-349.

Other diseases, such as myocarditis, congenital heart disease, cardiomyopathy, or myocardial infarction, also lead to RVF. As part of a low-pressure, low-resistance and highly distensible pulmonary vascular system, the right ventricle (RV) is poorly equipped, unlike the thick muscular wall of the left ventricle, to handle a large or rapid elevation of pulmonary artery pressure. See Jain V, Bordes S, Bhardwaj A. Physiology, Pulmonary Circulatory System. [Updated 2021 May 9]. In: StatPearls [Internet]. Treasure Island (FL): StatPearls Publishing; 2022 January-. Available from: https://www.ncbi.nlm.nih.gov/books/NBK525948. In early-stage PAH, the right ventricle increases contractility to accommodate the rise in afterload and to maintain cardiac output. See Wanner P M, Filipovic M. The Right Ventricle-You May Forget it, but It Will Not Forget You. J Clin Med. 2020; 9(2): 432. Published 2020 Feb. 5. doi: 10.3390/jem9020432. In more advanced stages, RV systolic function cannot keep pace with the increased afterload; decompensation progressively ensues with right heart chamber dilation and hypertrophy, leading to decreased cardiac output and overt signs of right heart failure e.g., dyspnea, pitting lower extremity edema, ascites, hepatic congestion and decreased systemic blood pressure. See Naeije R, Manes A. The right ventricle in pulmonary arterial hypertension. Eur Respir Rev. 2014 December; 23(134): 476-87.

The compensatory dilation and hypertrophy of the right ventricle, which attempts to maintain cardiac output at near normal levels is a sort of built-in self-defeating mechanism that ultimately reduces cardiac output; enlargement of the right ventricle displaces the interventricular septum into the left ventricle and impedes L V filling. See Gan C, Lankhaar J W, Marcus J T, Westerhof N, Marques K M, Bronzwaer J G, Boonstra A, Postmus P E, Vonk-Noordegraaf A. Impaired left ventricular filling due to right-to-left ventricular interaction in patients with pulmonary arterial hypertension. Am J Physiol Heart Circ Physiol. 2006; 290: H1528-H1533. This is termed interventricular dependence. Right ventricular dilation also leads to distortion of the tricuspid valve and significant tricuspid regurgitation, which further decreases right ventricular cardiac output. See Haddad F, Couture P, Tousignant C, et al. The right ventricle in cardiac surgery, a perioperative perspective: II. Pathophysiology, clinical importance, and management. Anesth Analg 2009; 108:422-433.

The World Health Organization (WHO) recognizes five classes of PH. Class I is PAH whereby the high blood pressure is between the lungs and the heart. Class II is PH due to left heart disease. Class II includes left ventricular systolic dysfunction, left ventricular diastolic dysfunction, valvular disease, congenital/acquired left heart inflow/outflow tract obstruction, and congenital cardiomyopathies. Class III is PH due to lung disease and/or hypoxia. Class III includes COPD, interstitial lung disease, pulmonary diseases with mixed restrictive and obstructive patterns, sleep disordered breathing, alveolar hyperventilation disorders, chronic high-altitude exposure, and developmental lung diseases. Class IV is chronic thromboembolic PH where blood clots in the lungs cause scar tissue that clogs and narrows blood vessels in the lungs. Class V is PH with unclear multifactorial mechanisms. Class V includes all PHs that cannot be classified as one of classes I, II, III, or IV.

Patients with PAH walk a careful clinical tightrope; clinical improvement depends on a negative fluid balance but over-diuresis may compromise the already low cardiac output and thereby further impair end-organ function. See Suri S S, Pamboukian S V. Optimal diuretic strategies in heart failure. Ann Transl Med. 2021; 9(6): 517. Currently, 10 PAH-specific therapies are approved, including endothelin receptor antagonists (ERA), phosphodiesterase-5 (PDE-5) inhibitors, and prostanoids, which target endothelial dysfunction and vasoconstriction. See Jacobs W, Vonk-Noordegraaf A. Epoprostenol in pulmonary arterial hypertension. Expert Opin Drug Metab Toxicol 2009; 5:83-90. See Montani D, Günther S, Dorfmüller P, Perros F, Girerd B, Garcia G, Jaïs X, Savale L, Artaud-Macari E, Price L C, Humbert M, Simonneau G, Sitbon O, Pulmonary arterial hypertension. Orphanet J Rare Dis. 2013 Jul. 6; 8:97. Nevertheless, because PAH remains a chronic and fatal disease, the search for better therapies that support right ventricular function continues.

### SUMMARY

Methods for treatment of pulmonary hypertension (PH) are provided. In some embodiments, a method for preventing or treating PH in a subject described herein comprises administering to the subject in need thereof an effective amount of RRx-001, or a pharmaceutically acceptable salt thereof. In some embodiments, the subject is a mammal subject. In some embodiments, the mammal subject is a human subject. In other embodiments, the mammal subject is a non-human subject. In some embodiments, the effective amount of RRx-001, or the pharmaceutically acceptable salt thereof, is a therapeutically effective amount of the RRx-001, or the pharmaceutically acceptable salt thereof.

In some embodiments, the method may further comprise administering an agent before, during, or after the administration of the effective amount of RRx-001, or the pharmaceutically acceptable salt thereof. In some embodiments, a method for treating PH in a patient is described herein that includes administering a therapeutically effective dose of RRx-001, or a pharmaceutically acceptable salt thereof, cither alone or in combination with another agent such as bosentan, epoprostenol or nitrite, and/or a blood product. In some embodiments, the therapeutically effective amount of the RRx-001, or the pharmaceutically acceptable salt thereof, is administered as a pharmaceutical composition comprising the other agent and/or the blood product.

The PH may be of World Health Organization (WHO) Class I, II, III, IV or V. In some embodiments, the PH may be selected from the group consisting of: congenital left to right intracardiac shunts, portal hypertension, persistent pulmonary hypertension of the newborn, collagen vascular diseases, HIV infection, drugs, and toxins. In some embodiments, the PH may be secondary to left heart disease, valvular heart disease, or restrictive cardiomyopathy (pulmonary venous hypertension). The most common causes of the PH may be mitral valve stenosis and left heart diastolic dysfunction. In some embodiments, the PH may be secondary to advanced chronic lung disease and environmental hypoxia. The advanced chronic lung disease and environmental hypoxia may include COPD, interstitial lung disease, sleep-disordered breathing, or alveolar hypoventilation disorder. In some embodiments, the PH may be secondary to at least one of chronic thrombotic disease and embolic disease. In some embodiments, the PH may be secondary to metabolic disorders, such as glycogen storage disease, thyroid disease, Gaucher disease, systemic diseases such as sarcoidosis, vasculitis, neurofibromatosis type 1, hematologic diseases such as myeloproliferative disorders as well as end-stage renal disease on dialysis, extrinsic compression of pulmonary vessels, or embolization of tumors.

The administering may include oral administration, transdermal administration, administration by inhalation, nasal administration, topical administration, intra-aural administration, rectal administration, intravenous administration, intravenous administration with a sample of autologous or allogenic blood from a compatible donor, intramuscular administration, subcutaneous administration, intraperitoneal administration, or combinations thereof. In some embodiments, administering the therapeutically effective amount of RRx-001, or the pharmaceutically acceptable salt thereof, is performed via a single administration.

In other embodiments, the administering the therapeutically effective amount of RRx-001, or the pharmaceutically acceptable salt thereof, is performed via at least two administrations. In some embodiments, administering the therapeutically effective amount of RRx-001, or the pharmaceutically acceptable salt thereof, is performed at a frequency of between about 1 time per hour and about 1 time per month. In some embodiments, administering the therapeutically effective amount of RRx-001, or the pharmaceutically acceptable salt thereof, is performed at a frequency of between about 4 times per day and about 1 time per week. In some embodiments, administering the therapeutically effective amount of RRx-001, or the pharmaceutically acceptable salt thereof, is performed at a frequency of between about 2 times per day and about 3 times per week. In some embodiments, administering the therapeutically effective amount of RRx-001, or the pharmaceutically acceptable salt thereof, is performed at a frequency of about one time a day.

The method may further include administering at least one other agent for treating the PH. The at least one other agent may include a nitrite, a PDE-5 inhibitor, a calcium channel blocker, a prostacyclin pathway agonist, an endothelin receptor antagonist, a diuretic, oxygen, a guanylate cyclase stimulator, an anti-coagulant, or a combination thereof. The PDE-5 inhibitor may be selected from the group consisting of: avanafil, lodenafil, mirodenafil, sildenafil, tadalafil, vardenafil, udenafil, zaprinast, and icariin. The prostacyclin pathway agonist may be selected from the group consisting of: poprostenol, treprostinil, iloprost, and selexipag. The endothelin receptor antagonist may be selected from the group consisting of: bosentan, macitentan, ambrisentan, sibotentan, sitaxsentan and tezosentan. The at least one other agent may include guanylate cyclase activator or riociguat.

In some embodiments, the blood product comprises erythrocyte cells. In some embodiments, the erythrocyte cells have not undergone any manipulation selected from the group consisting of genetic modification, electroporation, conjugation through biotin, conjugation to a cell-penetrating peptide, conjugation to hemoglobin, dimethyl sulfoxide osmotic pulse, endocytosis and hypotonic preswelling, hypotonic dilution, and hypo-osmotic dialysis. In some embodiments, the blood product is a mixture of packed red blood cells. In some embodiments, the blood product is whole blood. In some embodiments, the whole blood is autologous whole blood or donor-matched allogenic whole blood.

The treating (e.g., by administering the therapeutically effective amount of the RRx-001, or the pharmaceutically acceptable salt thereof) may result in an amelioration of at least one adverse outcome or event of the PH in the subject compared to non-treatment in the subject. Each adverse event of the at least one adverse event is selected from the group consisting of: dyspnea, exercise intolerance, systemic venous congestion, ascites and lower extremity edema. In some embodiments, the adverse outcomes or events of the PH may include cardiac remodeling. In some embodiments, the adverse outcomes or events of the PH may include pulmonary remodeling. In some embodiments, the adverse outcomes or events of the PH may include a decreased ejection fraction. In some embodiments, the probability of death in the subject may be reduced as a result of administering the therapeutically effective amount of the RRx-001 or the pharmaceutically acceptable salt. In some embodiments, the probability of hospitalization in the subject may be reduced as a result of administering the therapeutically effective amount of the RRx-001 or the pharmaceutically acceptable salt. In some embodiments, peripheral vasodilation may occur in the patient subsequent to administering the therapeutically effective amount of the RRx-001 or the pharmaceutically acceptable salt. In some embodiments, antioxidation may occur in the patient subsequent to administering the therapeutically effective amount of the RRx-001 or the pharmaceutically acceptable salt.

In some embodiments, the therapeutically effective dose (e.g., the therapeutically effective amount) of the RRx-001 or the pharmaceutically acceptable salt may be between about 1 mg and about 100 mg. In some embodiments, the therapeutically effective dose (e.g., the therapeutically effective amount) of the RRx-001 or the pharmaceutically acceptable salt may be between about 5 mg and about 50 mg. In some embodiments, the therapeutically effective dose (e.g., the therapeutically effective amount) of the RRx-001 or the pharmaceutically acceptable salt may be between about 10 mg and about 30 mg. In some embodiments, the therapeutically effective dose (e.g., the therapeutically effective amount) may be about 15 mg.

In some embodiments, the therapeutically effective dose (e.g., the therapeutically effective amount) of the RRx-001 or the pharmaceutically acceptable salt may be administered a frequency of between about 1 time per hour and about 1 time per month. In some embodiments, the therapeutically effective dose (e.g., the therapeutically effective amount) of the RRx-001 or the pharmaceutically acceptable salt may be administered a frequency of between about 4 times per day and about 1 time per week. In some embodiments, the therapeutically effective dose (e.g., the therapeutically effective amount) of the RRx-001 or the pharmaceutically acceptable salt may be administered a frequency of between about 2 times per day and about 3 times per week. In some embodiments, the therapeutically effective dose (e.g., the therapeutically effective amount) of the RRx-001 or the pharmaceutically acceptable salt may be administered a frequency of about 1 time per day.

In some embodiments, the treatment may result in an increased exercise tolerance compared to a baseline. In some embodiments, the treatment may result in decreased dyspnea compared to a baseline. In some embodiments, the effective amount of the RRx-001, or the pharmaceutically acceptable salt thereof, the at least one other agent, and/or the blood product may be administered at the same time. In some embodiments, the effective amount of the RRx-001, or the pharmaceutically acceptable salt thereof, the at least one other agent, and/or the blood product may be administered at different times. RRx-001 may be represented by the formula

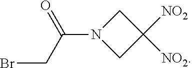 [SMILES: O=C(CBr)N1CC([N+](=O)[O-])([N+](=O)[O-])C1]

In some embodiments, a composition for preventing or treating the PH in the subject in need thereof is disclosed herein. The composition comprises an effective amount of RRx-001, or a pharmaceutically acceptable salt thereof, and at least one pharmaceutically acceptable carrier or excipient. In some embodiments, the effective amount of RRx-001, or the pharmaceutically acceptable salt thereof, is the therapeutically effective amount of the RRx-001, or the pharmaceutically acceptable salt thereof. In some embodiments, the composition further comprises an agent. In some embodiments, the agent is an agent for treating PH. In some embodiments, the composition additionally or alternatively comprises the blood product.

### BRIEF DESCRIPTION OF THE DRAWINGS

FIG. 1A is a plot of a distribution of collected data of mean pulmonary artery pressure (mPAP) of male Wistar rats from various groups of a study.

FIG. 1B is a plot of a distribution showing mean aortic pressure (mAP) of male Wistar rats from various groups of the same study.

FIG. 2 shows a graph of collected data of a wet weight ratio of a right ventricle (RV) against a left ventricle (LV) plus septum (SP), RV/(LV+SP).

FIG. 3A is a graph of a distribution of collected data of an ejection fraction based on measurements from an echocardiograph.

FIG. 3B is a graph of a distribution of collected data of fractional shortening based on measurements from the echocardiograph.

FIG. 4A is a graph of a distribution of collected data showing lung morphological changes of medial wall thickness of removed lungs from male Wistar rats relative to external diameter.

FIG. 4B is a graph of a distribution of collected data showing lung morphological changes of medial wall area relative to lumen area of removed lungs from male Wistar rats.

FIG. 5A is a graph of a distribution of collected data of MPAP of male Wistar rats from various groups of a study that included administration of nitrite.

FIG. 5B is a graph of a distribution of collected data of mAP of male Wistar rats from various groups of a study that included administration of nitrite.

FIG. 6A is a graph of a distribution of collected data of mPAP based on nitrite dose of male Wistar rats from various groups of a study that included administration of nitrite.

FIG. 6B is a graph of a distribution of collected data of mAP based on nitrite dose of male Wistar rats from various groups of a study that included administration of nitrite.

FIG. 7 shows a graph of collected data of a wet weight ratio of RV/(LV+SP) from various groups of a study that included administration of nitrite.

FIG. 8 shows a graph of collected data of a wet weight ratio of RV/(LV+SP) based on nitrite dose from various groups of a study that included administration of nitrite.

FIG. 9 shows a graph of a distribution of collected data of methemoglobin (metHb) in the blood of Wistar rats.

FIG. 10 shows representative images showing hematoxylin and eosin (H&E) staining in the pulmonary arteriole of male Wistar rats.

### DETAILED DESCRIPTION

The disclosed subject matter is a composition and method of treatment for pulmonary hypertension with RRx-001. The treatment is based in part on a trial on male Wistar rats where RRx-001 was found to be effective in treating pulmonary hypertension.

The terms “a” and “an” as used herein mean “one or more” and include the plural unless the context is inappropriate.

Reference to “about” a value or parameter herein includes (and describes) variations that are directed to that value or parameter per se. In embodiments, the term “about” refers to +/−10%, +/−5%, or +/−1%, of the designated value.

The “comprise” or variations such as “comprises” or “comprising,” will be understood to imply the inclusion of a stated element, integer or step, or group of elements, integers or steps, but not the exclusion of any other element, integer or step, or group of elements, integers or steps. Embodiments described herein also include “consisting” and/or “consisting essentially of” aspects.

“Treatment”, “treating” or similar phrases refer to obtaining beneficial or desired results, such as clinical results, for a subject, suffering from the PH or a condition associated with the PH. As used herein, the term “treating” includes any effect, e.g., lessening, reducing, modulating, ameliorating or eliminating, that results in the improvement of the condition, disorder, and the like, or ameliorating a symptom thereof. Beneficial or desired results include any one or more of: alleviating one or more symptoms of the disorder, diminishing the extent of the disorder, delaying or slowing the disorder progression, and improving quality of life.

As used herein, the term “effective amount” refers to the amount of a compound (e.g., a compound of the present invention) sufficient to effect beneficial or desired results. An effective amount can be administered in one or more administrations, applications or dosages and is not intended to be limited to a particular formulation or administration route.

As used herein, the term “subject” or “patient” refers to an organism to be treated by the methods of the present invention. Such organisms are preferably mammals (e.g., murines, simians, equines, bovines, porcines, canines, felines, and the like), and more preferably humans.

The phrase “parenteral administration” and “administered parenterally” as used herein means modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal, and intrasternal injection and infusion.

As used herein, the term “composition” or “pharmaceutical composition” refers to the combination of an active agent with an excipient or a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants. (See e.g., Martin, Remington's Pharmaceutical Sciences, 15th Ed., Mack Publ. Co., Easton, PA [1975]).

As used herein, the term “pharmaceutically acceptable salt” refers to any circular salt (e.g., acid or base) of a compound of the present invention suitable for pharmaceutical administration which, upon administration to a subject, is capable of providing a compound of this invention or an active metabolite or residue thereof. As is known to those of skill in the art, “salts” of the compounds of the present invention may be derived from inorganic or organic acids and bases.

Examples of acids include, but are not limited to, hydrochloric, hydrobromic, sulfuric, nitric, perchloric, fumaric, maleic, phosphoric, glycolic, lactic, salicylic, succinic, toluene psulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metals (e.g., sodium) hydroxides, alkaline earth metals (e.g., magnesium), hydroxides, ammonia, and compounds of formula NW4+, wherein W is C1-4 alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH⁴⁺, and NW⁴⁺ (wherein W is a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

RRx-001 (also called ABDNAZ), with the chemical name 2-bromo-1-(3,3-dinitroazetidin-1-yl) ethan-1-one, is a small cyclic nitro compound. RRx-001 is a minimally toxic anticancer agent in Phase 3 clinical trials for the treatment of cancer. RRx-001 has the following structure:

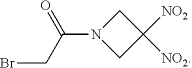 [SMILES: O=C(CBr)N1CC([N+](=O)[O-])([N+](=O)[O-])C1]

An exemplary embodiment of the disclosed subject matter comprises treating the PH where the PH is any of the five classes of the PH as defined by the World Health Organization, which are: class I—PAH, class II—PH due to left heart disease, class III—PH due to lung disease and/or hypoxia, class IV-chronic thromboembolic pulmonary hypertension (CTEPH), and class V—PH with unclear multifactorial mechanisms.

In various embodiments, the disclosed subject matter comprises treatment of idiopathic PAH or familial PAH. In various embodiments, the PAH is associated with or secondary to a collagen vascular disease, a congenital heart disease, portal hypertension, or HIV infection. In various embodiments, the PAH is associated with or secondary to ingestion of a drug or toxin, hereditary hemorrhagic telangiectasia, splenectomy, pulmonary veno-occlusive disease (PVOD), or pulmonary capillary hemangiomatosis (PCH). In various embodiments, the PAH is associated with or secondary to PAH with significant venous or capillary involvement or CTEPH.

In various embodiments, the disclosed subject matter comprises the PH, wherein the PH is associated with or secondary to a condition including congenital left to right intracardiac shunts, persistent pulmonary hypertension of the newborn, restrictive cardiomyopathy (pulmonary venous hypertension), mitral valve stenosis, left heart diastolic dysfunction, chronic thrombotic disease or embolic disease.

In various embodiments, the disclosed subject matter comprises the PH, wherein the PH is associated with or secondary to a condition including advanced chronic lung disease or environmental hypoxia, chronic obstructive pulmonary disease (COPD), interstitial lung disease, sleep-disordered breathing, or alveolar hypoventilation disorder.

In various embodiments, the disclosed subject matter comprises the PH, wherein the PH is associated with or secondary to a condition including glycogen storage disease, thyroid disease, Gaucher disease, systemic diseases such as sarcoidosis, vasculitis, neurofibromatosis type 1, hematologic diseases such as myeloproliferative disorders as well as end-stage renal disease on dialysis, extrinsic compression of pulmonary vessels, or embolization of tumors.

### Methods of Administration

The RRx-001, or a pharmaceutically acceptable salt thereof, may be administered to a human patient or animal subject in a variety of embodiments. In various embodiments, the RRx-001, or a pharmaceutically acceptable salt thereof, is administered via inhalation, nasal administration, topical administration, oral administration, transdermal administration, intra-aural administration, rectal administration, intravenous administration, intramuscular administration, subcutaneous administration, intraperitoneal administration, or combinations thereof.

An example of oral administration comprises the human patient or animal subject taking the RRx-001, or a pharmaceutically acceptable salt thereof, by mouth. Transdermal administration comprises administering the RRx-001 through the skin of the human patient or the animal subject. An example of transdermal administration includes applying a composition containing the RRx-001, or a pharmaceutically acceptable salt thereof, that permeates the skin and delivers the RRx-001. Intra-aural administration comprises administering the RRx-001, or a pharmaceutically acceptable salt thereof, through the ear. An example of intra-aural administration may include ear drops for the human patient or the animal subject that contain an amount of the RRx-001, or a pharmaceutically acceptable salt thereof. Rectal administration comprises administration of the RRx-001, or a pharmaceutically acceptable salt thereof, through the anus of the human patient or the animal subject. An example of rectal administration comprises administering a suppository that contains an amount of the RRx-001 to the human patient or the animal subject.

Intravenous administration comprises injecting the RRx-001, or a pharmaceutically acceptable salt thereof, directly into a vein of the human patient or the animal subject and may include a combination with a sample of autologous or allogenic blood from a compatible donor. Intramuscular administration comprises injecting a composition that includes the RRx-001, or a pharmaceutically acceptable salt thereof, directly into a muscle of the human patient or the animal subject. Subcutaneous administration comprises delivering the RRx-001, or a pharmaceutically acceptable salt thereof, just under a layer of skin. An example of subcutaneous administration comprises injecting a composition including the RRx-001, or a pharmaceutically acceptable salt thereof, into a layer a fatty tissue just under the skin of the human patient or the animal subject. Intraperitoneal administration comprises injecting the RRx-001, or a pharmaceutically acceptable salt thereof, into the peritoneum of the human patient or the animal subject.

In various embodiments, the RRx-001, or a pharmaceutically acceptable salt thereof, is administered in combination with one or more other agents. The term “in combination” when used herein, may refer to administering two or more agents concurrently or in sequence. Concurrent administration may refer to administering two agents at approximately the same time. Concurrent administration may refer to administering an agent at regular intervals where the regular intervals overlap with administration of one or more other agents. Sequential administration may refer to administering two or more agents at different times, but such that the two or more agents result in a combined effect on the human patient or the animal subject.

In various embodiments, the other agent for treatment of the PH, which is administered in addition to the RRx-001, or a pharmaceutically acceptable salt thereof, and includes at least one of a nitrite, a PDE-5 inhibitor, a calcium channel blocker, a prostacyclin pathway agonist, an endothelin receptor antagonist, a diuretic, oxygen, a guanylate cyclase stimulator, or an anti-coagulant. The PDE-5 inhibitor may include at least one of avanafil, lodenafil, mirodenafil, sildenafil, tadalafil, vardenafil, udenafil, zaprinast, or icariin. The prostacyclin pathway agonist, which is administered in combination with the RRx-001, or a pharmaceutically acceptable salt thereof, may include at least one of epoprostenol, treprostinil, iloprost, or selexipag. In various embodiments, the endothelin receptor antagonist, which is administered in combination with the RRx-001, may include at least one of bosentan, macitentan, ambrisentan, sibotentan, sitaxsentan or tezosentan. In various embodiments, the other agent that is administered in combination with the RRx-001 for treatment of pulmonary hypertension includes guanylate cyclase activator or riociguat.

In various embodiments, symptoms of the PH are ameliorated subsequent to administration of the RRx-001, or a pharmaceutically acceptable salt thereof, with or without another agent. The term “ameliorated”, as used herein, may refer to alleviating or lessening one or more symptoms of the type of PH that the human patient or the animal subject has. The term “ameliorated”, as used herein, may refer to alleviating or lessening one or more symptoms relative to non-treatment of any type of PH.

In various embodiments, at least one of the symptoms of the PH includes oxidative stress. In various embodiments, a least one of the symptoms of the PH includes cardiac remodeling. In various embodiments, a least one of the symptoms of the PH includes pulmonary remodeling. In various embodiments, a probability of death is reduced in the human patient or the animal subject subsequent to administration of the RRx-001, or a pharmaceutically acceptable salt thereof. In various embodiments, a probability of hospitalization is reduced subsequent to administration of the RRx-001, or a pharmaceutically acceptable salt thereof, in the human patient or the animal subject. The term, “hospitalization” may refer to the human patient or the animal subject being admitted to a hospital for care related to symptoms for the PH. The term “hospitalization”, as used herein may refer to the human patient or the animal subject receiving care from a medical professional, such as a nurse or doctor, at least for symptoms of the PH.

In various embodiments, peripheral vasodilation occurs in the human patient or the animal subject subsequent to administration of the RRx-001, or a pharmaceutically acceptable salt thereof. Peripheral vasodilation, as used herein, may refer to widening of one or more blood vessels.

In some embodiments, a subject is a neonate (i.e., less than one month old), an infant (at least one month to one year old) or a toddler (one year to three years old). In other embodiments, a patient is a child (three to 18 years old). In still other embodiments, the subject is an adult (≥18 years old).

### Amount of RRX-001

In various embodiments, the RRx-001 may be administered as RRx-001, or a pharmaceutically acceptable salt thereof. The RRx-001 may be administered in various amounts. The dosages provided herein refer to the amount of the RRx-001, excluding the weight of any counterion that may be present. The amount may refer to a total amount of the RRx-001 that is administered over a period of time or a dosage of individual administrations of the RRx-001. In various embodiments, the RRx-001 is administered in doses of between about 1 mg and 500 mg. In various embodiments, the RRx-001 is administered in doses of between about 0.5 mg and 250 mg. In various embodiments, the RRx-001 is administered in doses of between about 1 mg and 2 mg. In various embodiments, the RRx-001 is administered in doses of between about 2 mg and 3 mg. In various embodiments, the RRx-001 is administered in doses of between about 3 mg and 4 mg. In various embodiments, the RRx-001 is administered in doses of between about 4 mg and 5 mg. In various embodiments, the RRx-001 is administered in doses of between about 5 mg and 6 mg. In various embodiments, the RRx-001 is administered in doses of between about 6 mg and 7 mg. In various embodiments, the RRx-001 is administered in doses of between about 7 mg and 8 mg. In various embodiments, the RRx-001 is administered in doses of between about 8 mg and 9 mg. In various embodiments, the RRx-001 is administered in doses of between about 9 mg and 10 mg. In various embodiments, the RRx-001 is administered in doses of between about 10 mg and 12 mg. In various embodiments, the RRx-001 is administered in doses of between about 12 mg and 14 mg. In various embodiments, the RRx-001 is administered in doses of between about 14 mg and 16 mg. In various embodiments, the RRx-001 is administered in doses of between about 16 mg and 18 mg. In various embodiments, the RRx-001 is administered in doses of between about 18 mg and 20 mg. In various embodiments, the RRx-001 is administered in doses of between about 20 mg and 22 mg. In various embodiments, the RRx-001 is administered in doses of between about 22 mg and 24 mg. In various embodiments, the RRx-001 is administered in doses of between about 24 mg and 26 mg. In various embodiments, the RRx-001 is administered in doses of between about 26 mg and 28 mg. In various embodiments, the RRx-001 is administered in doses of between about 28 mg and 30 mg. In various embodiments, the RRx-001 is administered in doses of between about 30 mg and 35 mg. In various embodiments, the RRx-001 is administered in doses of between about 35 mg and 40 mg. In various embodiments, the RRx-001 is administered in doses of between about 40 mg and 45 mg. In various embodiments, the RRx-001 is administered in doses of between about 45 mg and 50 mg. In various embodiments, the RRx-001 is administered in doses of between about 50 mg and 55 mg. In various embodiments, the RRx-001 is administered in doses of between about 55 mg and 60 mg. In various embodiments, the RRx-001 is administered in doses of between about 60 mg and 65 mg. In various embodiments, the RRx-001 is administered in doses of between about 65 mg and 70 mg. In various embodiments, the RRx-001 is administered in doses of between about 70 mg and 75 mg. In various embodiments, the RRx-001 is administered in doses of between about 75 mg and 80 mg. In various embodiments, the RRx-001 is administered in doses of between about 80 mg and 85 mg. In various embodiments, the RRx-001 is administered in doses of between about 85 mg and 90 mg. In various embodiments, the RRx-001 is administered in doses of between about 90 mg and 95 mg. In various embodiments, the RRx-001 is administered in doses of between about 95 mg and 100 mg. In various embodiments, the RRx-001 is administered in doses of between about 100 mg and 105 mg. In various embodiments, the RRx-001 is administered in doses of between about 105 mg and 110 mg. In various embodiments, the RRx-001 is administered in doses of between about 110 mg and 115 mg. In various embodiments, the RRx-001 is administered in doses of between about 115 mg and 120 mg. In various embodiments, the RRx-001 is administered in doses of between about 120 mg and 125 mg. In various embodiments, the RRx-001 is administered in doses of between about 125 mg and 130 mg. In various embodiments, the RRx-001 is administered in doses of between about 130 mg and 135 mg. In various embodiments, the RRx-001 is administered in doses of between about 135 mg and 140 mg. In various embodiments, the RRx-001 is administered in doses of between about 140 mg and 145 mg. In various embodiments, the RRx-001 is administered in doses of between about 145 mg and 150 mg. In various embodiments, the RRx-001 is administered in doses of between about 150 mg and 155 mg. In various embodiments, the RRx-001 is administered in doses of between about 155 mg and 160 mg. In various embodiments, the RRx-001 is administered in doses of between about 160 mg and 165 mg. In various embodiments, the RRx-001 is administered in doses of between about 165 mg and 170 mg. In various embodiments, the RRx-001 is administered in doses of between about 170 mg and 175 mg. In various embodiments, the RRx-001 is administered in doses of between about 175 mg and 180 mg. In various embodiments, the RRx-001 is administered in doses of between about 180 mg and 185 mg. In various embodiments, the RRx-001 is administered in doses of between about 185 mg and 190 mg. In various embodiments, the RRx-001 is administered in doses of between about 190 mg and 195 mg. In various embodiments, the RRx-001 is administered in doses of between about 195 mg and 200 mg.

### Frequency of Administration

The amount of the RRx-001, or a pharmaceutically acceptable salt thereof, may be administered at various intervals, which are referred to herein as a frequency of administration. In various embodiments, the frequency of administration of the RRx-001, or a pharmaceutically acceptable salt thereof, may be between about one dose per hour and about 1 dose every 6 months. In various embodiments, the frequency of administration of the RRx-001, or a pharmaceutically acceptable salt thereof, may be between about one dose every 2 hours and about 1 dose every 4 months. In various embodiments, the frequency of administration of the RRx-001, or a pharmaceutically acceptable salt thereof, may be between about one dose every 4 hours and about 1 dose every 3 months. In various embodiments, the frequency of administration of the RRx-001 may be between about one dose every 4 hours and about 1 dose every 3 months. In various embodiments, the frequency of administration of the RRx-001, or a pharmaceutically acceptable salt thereof, may be between about one dose every 6 hours and about 1 dose every 2 months. In various embodiments, the frequency of administration of the RRx-001, or a pharmaceutically acceptable salt thereof, may be between about one dose every 10 hours and about 1 dose every 6 weeks. In various embodiments, the frequency of administration of the RRx-001, or a pharmaceutically acceptable salt thereof, may be between about two doses every day and about 1 dose every 6 weeks. In various embodiments, the frequency of administration of the RRx-001, or a pharmaceutically acceptable salt thereof, may be between about 2 doses every day and about 1 dose every 6 weeks. In various embodiments, the frequency of administration of the RRx-001, or a pharmaceutically acceptable salt thereof, may be between about 1 dose every day and about 1 dose every 4 weeks. In various embodiments, the frequency of administration of the RRx-001, or a pharmaceutically acceptable salt thereof, may be between about 4 doses every week and about 1 dose every 3 weeks. In various embodiments, the frequency of administration of the RRx-001, or a pharmaceutically acceptable salt thereof, may be between about 3 doses every week and about 1 dose every 2 weeks. In various embodiments, the frequency of administration of the RRx-001, or a pharmaceutically acceptable salt thereof, may be between about 2 doses every week and about 1 dose every week. In various embodiments, the frequency of administration of the RRx-001, or a pharmaceutically acceptable salt thereof, may be about 1 dose per day.

In various embodiments, the frequency of administration of the RRx-001, or a pharmaceutically acceptable salt, may be between about one dose per 30 min and about 1 dose per hour. In various embodiments, the frequency of administration of the RRx-001, or a pharmaceutically acceptable salt thereof, may be between about one dose per hour and about 1 dose per 2 hours. In various embodiments, the frequency of administration of the RRx-001, or a pharmaceutically acceptable salt thereof, may be between about one dose per 2 hours and about 1 dose per 4 hours. In various embodiments, the frequency of administration of the RRx-001, or a pharmaceutically acceptable salt thereof, may be between about one dose per 4 hours and about 1 dose per 6 hours. In various embodiments, the frequency of administration of the RRx-001, or a pharmaceutically acceptable salt thereof, may be between about one dose per 6 hours and about 1 dose per 8 hours. In various embodiments, the frequency of administration of the RRx-001, or a pharmaceutically acceptable salt thereof, may be between about one dose per 8 hours and about 1 dose per 12 hours. In various embodiments, the frequency of administration of the RRx-001, or a pharmaceutically acceptable salt thereof, may be between about one dose per 12 hours and about 1 dose per 16 hours. In various embodiments, the frequency of administration of the RRx-001, or a pharmaceutically acceptable salt thereof, may be between about one dose per 16 hours and about 1 dose per 20 hours. In various embodiments, the frequency of administration of the RRx-001, or a pharmaceutically acceptable salt thereof, may be between about one dose per 20 hours and about 1 dose per 24 hours. In various embodiments, the frequency of administration of the RRx-001, or a pharmaceutically acceptable salt thereof, may be between about 1 dose per day and about 1 dose per 2 days. In various embodiments, the frequency of administration of the RRx-001, or a pharmaceutically acceptable salt thereof, may be between about 1 dose per 2 days and about 1 dose per 3 days. In various embodiments, the frequency of administration of the RRx-001, or a pharmaceutically acceptable salt thereof, may be between about 1 dose per 3 days and about 1 dose per 5 days. In various embodiments, the frequency of administration of the RRx-001, or a pharmaceutically acceptable salt thereof, may be between about 1 dose per 5 days and about 1 dose per week. In various embodiments, the frequency of administration of the RRx-001, or a pharmaceutically acceptable salt thereof, may be between about 1 dose per week and about 1 dose per 2 weeks. In various embodiments, the frequency of administration of the RRx-001, or a pharmaceutically acceptable salt thereof, may be between about 1 dose per 2 weeks and about 1 dose per 3 weeks. In various embodiments, the frequency of administration of the RRx-001, or a pharmaceutically acceptable salt thereof, may be between about 1 dose per 3 weeks and about 1 dose per 4 weeks. In various embodiments, the frequency of administration of the RRx-001, or a pharmaceutically acceptable salt thereof, may be between about 1 dose per 4 weeks and about 1 dose per 6 weeks. In various embodiments, the frequency of administration of the RRx-001, or a pharmaceutically acceptable salt thereof, may be between about 1 dose per 8 weeks and about 1 dose per 12 weeks.

### Pharmaceutical Composition

The present disclosure provides compositions or pharmaceutical compositions for preventing or treating the PH in a subject in need thereof. As a general matter, the pharmaceutical composition contains at least one active agent and a pharmaceutically acceptable carrier. In some embodiments, the pharmaceutical compositions of the present invention may be specially formulated for administration in solid or liquid form, including those adapted for the following: (1) oral administration, for example, drenches (aqueous or non-aqueous solutions or suspensions), tablets (e.g., those targeted for buccal, sublingual, and/or systemic absorption), boluses, powders, granules, pastes for application to the tongue; (2) parenteral administration by, for example, subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the present invention include those suitable for oral, nasal, topical (including buccal and sublingual), and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders a compound of the present invention orally bioavailable.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

In certain embodiments, it may be desirable to introduce the compositions disclosed herein into the central nervous system by any suitable route, including intraventricular, intrathecal and epidural injection. Intraventricular injection may be facilitated by an intraventricular catheter, for example, attached to a reservoir, such as an Ommaya reservoir.

In some embodiments, the pharmaceutical composition is configured as an inhalable formulation. In some embodiments, the inhalable formulation is configured as a dosage form adapted for pulmonary or nasal administration to the subject. In some embodiments, for example, dosage forms may include those adapted for inhalation such as aerosols and dry powders. In some embodiments, the formulation described herein is suitable for topical delivery to the lung via nose inhalation and/or mouth inhalation. In other embodiments, the compositions disclosed herein may also be administered directly to the lung by inhalation by a number of different devices.

In some embodiments, the inhalable formulation is configured as an aerosol formulation that comprises a propellant. In some embodiments, the propellant can provide energy to deliver molecules of any of the compounds described herein to the lung. Representative propellants are disclosed in U.S. Pat. No. 6,932,962 B1 and U.S. Pat. No. 8,367,734 B1. In some embodiments, the propellant is presented in the aerosol formulation in an amount ranging from 98% to 99% (w/w) relative to the total weight of the aerosol formulation.

In some embodiments, the aerosol formulation further comprises a surfactant, a co-solvent, and/or a pH buffer. The surfactant can give fine dispersions of the compounds described herein in the propellant and can stabilize the mixture of the compounds described herein in the propellant. In some embodiments, the surfactant comprises a fatty acid or a pharmaceutically acceptable salt thereof, a bile salt, a phospholipid, or an alkyl saccharide. In some embodiments, the surfactant is presented in the formulations described herein in an amount of less than 5% (w/w) (e.g., less than 4%, less than 3%, less than 2%, less than 1% by weight) relative to the total weight of the aerosol formulation.

In some embodiments, the co-solvent can help to stabilize the surfactant and improve the dispersion characteristics. In some embodiments, exemplary co-solvents include ethyl alcohol, isopropyl alcohol, propylene glycol, ethylene glycol, propane, butane, isobutane, pentane, dimethyl ether, diethyl ether and the like. In some embodiments, the co-solvent is present in the formulation in an amount ranging from 0.5% to 20% w/w of the total weight of the formulation. In some embodiments, the co-solvent is present in the formulation in an amount ranging from 0.5% to 5% w/w of the total weight of the formulation. In some embodiments, the co-solvent is present in the formulation in an amount ranging from 0.5% to 1.5% (w/w) of the total weight of the formulation. Representative surfactants, co-solvents, and pH buffers are disclosed in U.S. Pat. No. 6,932,962 B1 and U.S. Pat. No. 8,367,734 B1.

In some embodiments, provided herein are combinations containing the aerosol formulation with the propellant and a pressurized bottle or a nebulizer. In some embodiments, the aerosol formulation with the propellant may be packed in pressurized bottles, where a dosage controller may be used with the pressurized bottle to control the amount of drug being administrated in each spray. In some embodiments, the aerosol formulation with the propellant may be packed in pressurized bottles with a dosage controller, where the dosage controller comprises a valve that controls the delivery of a metered amount of the drug.

In some embodiments, the aerosol formulation is propellant-free and comprises the effective amount of the RRx-001 or the pharmaceutical composition and a solvent. In some embodiments, exemplary solvents include water and alcohols, such as ethanol, isopropanol, and glycols, such as propylene glycol, polyethylene glycol, polypropylene glycol, glycol ether, glycerol and polyoxyethylene alcohols. In some embodiments, the solvent is present in the propellant-free aerosol formulation in an amount ranging from about 0.01% to about 90% (w/w), or about 0.01% to about 50% (w/w), or about 0.01% to about 25% (w/w), or about 0.01% to about 10% (w/w), or about 0.01% to about 5% (w/w) relative to the total weight of the aerosol formulation.

In some embodiments, the propellant-free aerosol formulation may further comprise an emulsifying agent. In some embodiments, exemplary emulsifying agents are disclosed in U.S. Pat. No. 9,498,437 B2. In some embodiments, the emulsifying agent is present in the propellant-free aerosol formulations in an amount ranging from about 0.001% to about 50% (w/w), or about 0.001% to about 25% (w/w), or about 0.001% to about 10% (w/w), or about 0.001% to about 2% (w/w), or about 0.001% to about 1% (w/w) relative to the total weight of the aerosol formulation.

In some embodiments, the propellant-free aerosol formulation may further comprise a complexing agent. In some embodiments, exemplary complexing agents include, but are not limited to, ethylenediaminetetraacetic acid (EDTA) or a pharmaceutically acceptable salt thereof, such as the disodium salt, citric acid, nitrilotriacetic acid and the salts thereof, and sodium edetate. Representative complexing agents are disclosed in U.S. Pat. No. 9,498,437 B2. In some embodiments, the complexing agent is present in the propellant-free aerosol formulations in an amount ranging from about 0.001% to about 50% (w/w), or about 0.001% to about 25% (w/w), or about 0.001% to about 10% (w/w), or about 0.001% to about 2% (w/w), or about 0.001% to about 1% (w/w) relative to the total weight of the aerosol formulation.

In some embodiments, the propellant-free aerosol formulation may further comprise a tonicity agent that can adjust the isotonicity of the present formulations. In some embodiments, exemplary tonicity agents include, but are not limited to, sodium chloride, potassium chloride, zinc chloride, calcium chloride or mixtures thereof. Other osmotic adjusting agents may also include, but are not limited to, mannitol, glycerol, and dextrose or mixtures thereof. Representative tonicity agents are disclosed in U.S. Pat. No. 9,498,437 B2. In some embodiments, the tonicity agent is present in the propellant-free aerosol formulations in an amount ranging from about 0.01% to about 10% (w/w), or about 1% to about 10% (w/w), or about 1% to about 6% (w/w) relative to the total weight of the aerosol formulation. In some embodiments, the aerosol formulation may further comprise the pH buffer.

In some embodiments, provided herein are combinations containing the propellent-free aerosol formulation provided herein and a nebulizer. In some embodiments, the nebulizer can nebulize liquid formulations, including the propellant-free aerosol formulations detailed herein, and produce a nebulized aerosol mist. In some embodiments, the nebulizer may further have an internal baffle, which can selectively remove large droplets from the mist by impaction and allow the droplets to return to the reservoir, so that only fine aerosol droplets are entrained into the lung of the subject by the inhaling air/oxygen. Examples of nebulizers include devices supplied by Sheffield Pharmaceuticals, St. Louis, MO. (Armer et al., U.S. Pat. No. 5,954,047; van der Linden et al., U.S. Pat. No. 5,950,619; van der Linden et al., U.S. Pat. No. 5,970,974) and Batelle Pulmonary Therapeutics, Columbus, OH).

In some embodiments, a Metered Dose Inhaler (“MDI”), which utilizes canisters that contain a suitable low boiling propellant, (e.g., dichlorodifluoromethane, trichlorofluoromethane, dichlorotetrafluoroethane, carbon dioxide or any other suitable gas) may be used to deliver the RRx-001 and/or pharmaceutical compositions thereof directly to the lung. Specifically, the MDI comprises an aerosol container suitable for containing a propellant-based aerosol formulation and/or a metering valve, for example a side valve, which controls the release of the aerosol formulation to the subject. Representative methods and devices to administer the aerosol formulation with the propellant are disclosed in U.S. Pat. No. 9,498,437 B2.

In another embodiment, a Dry Powder Inhaler (“DPI”) device may be used to administer the compositions disclosed herein to the lung. DPI devices typically use a mechanism such as a burst of gas to create a cloud of dry powder inside a container, which may then be inhaled by the patient and are well known in the art. In a particular embodiment, a popular variation is the multiple dose DPI (“MDDPI”) system, which allows for the delivery of more than one therapeutic dose. MDDPI devices are commercially available from a number of pharmaceutical companies e.g., Schering Plough, Madison, NJ). For example, capsules and cartridges of gelatin for use in an inhaler or insufflator may be formulated containing a powder mix of the compositions disclosed herein and a suitable powder base such as lactose or starch for these systems.

In some embodiments, another type of device that may be used to deliver the compositions disclosed herein to the lung is a liquid spray device supplied, for example, by Aradigm Corporation, Hayward, CA. Liquid spray systems use extremely small nozzle holes to aerosolize liquid drug formulations that may then be directly inhaled into the lung.

In some embodiments, a nebulizer is used to deliver the compositions disclosed herein to the lung. Nebulizers create aerosols from liquid drug formulations by using, for example, ultrasonic energy to form fine particles that may be readily inhaled (see e.g., Verschoyle et al., British J. Cancer, 1999, 80, Suppl. 2, 96). Examples of nebulizers include devices supplied by Sheffield Pharmaceuticals, St. Louis, MO. (Armer et al., U.S. Pat. No. 5,954,047; van der Linden et al., U.S. Pat. No. 5,950,619; van der Linden et al., U.S. Pat. No. 5,970,974) and Batelle Pulmonary Therapeutics, Columbus, OH).

In other embodiments, an electrohydrodynamic (“EHD”) aerosol device is used to deliver the compositions disclosed herein to the lung of a patient. EHD aerosol devices use electrical energy to aerosolize liquid drug solutions or suspensions (see e.g., Noakes et al., U.S. Pat. No. 4,765,539). The electrochemical properties of the formulation may be important parameters to optimize when delivering the RRx-001 and/or pharmaceutical composition thereof to the lung with an EHD aerosol device. EHD aerosol devices may more efficiently deliver drugs to the lung than existing pulmonary delivery technologies.

Suitable pharmaceutically acceptable excipients will vary depending upon the particular dosage form chosen. In some embodiments, for example, certain pharmaceutically acceptable excipients may be chosen for their ability to: facilitate the production of aerosol for inhalation, facilitate the production of solution or mist for inhalation, facilitate the production of dry powder for inhalation, or facilitate the production of stable dosage forms.

In some embodiments, the compositions disclosed herein can be delivered via sustained release systems, e.g., oral sustained release systems. In other embodiments, a pump may be used (e.g., Langer, supra, Sefton, 1987, CRC Crit. Ref Biomed. Eng. 14:201; Saudek et al., 1989, N. Engl. J Med. 321:574).

In some embodiments, polymeric materials can be used (e.g., “Medical Applications of Controlled Release,” Langer and Wise (eds.), CRC Press, Boca Raton, Florida (1974); “Controlled Drug Bioavailability,” Drug Product Design and Performance, Smolen and Ball (eds.), Wiley, New York (1984); Ranger et al., 1983, J Macromol. Sci. Rev. Macromol Chem. 23:61; Levy et al., 1985, Science 228:190; During et al., 1989, Ann. Neurol. 25:351; Howard et al., 1989, J. Neurosurg. 71:105).

In other embodiments, polymeric materials are used for oral sustained release delivery. Polymers include, but are not limited to, sodium carboxymethylcellulose, hydroxypropylcellulose, hydroxypropylmethylcellulose and hydroxyethylcellulose (most preferred, hydroxypropyl methylcellulose). Other cellulose ethers have been described (Alderman, Int. J. Pharm. Tech. & Prod. Mfr. 1984, 5(3) 1-9). Factors affecting drug release are well known to the skilled artisan and have been described in the art (Bamba et al., Int. J. Pharm. 1979, 2, 307).

In other embodiments, enteric-coated preparations can be used for oral sustained release administration. Coating materials include polymers with a pH-dependent solubility (i.e., pH-controlled release), polymers with a slow or pH-dependent rate of swelling, dissolution or erosion (i.e., time-controlled release), polymers that are degraded by enzymes (i.e., enzyme-controlled release) and polymers that form firm layers that are destroyed by an increase in pressure (i.e., pressure-controlled release).

In other embodiments, osmotic delivery systems are used for oral sustained release administration (Verma et al., Drug Dev. Ind. Pharm., 2000, 26:695-708). In some embodiments, OROS™ osmotic devices are used for oral sustained release delivery devices (Theeuwes et al., U.S. Pat. No. 3,845,770; Theeuwes et al., U.S. Pat. No. 3,916,899).

In yet other embodiments, a controlled-release system can be placed in proximity of the target of RRx-001 described herein and/or pharmaceutical composition, thus requiring only a fraction of the systemic dose (e.g., Goodson, in “Medical Applications of Controlled Release,” supra, vol. 2, pp. 115-138 (1984)). Other controlled-release systems previously may also be used (Langer, 1990, Science 249:1527-1533).

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

In some cases, in order to prolong the effect of a drug, it is desirable to slow the absorption of the drug administered by subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

When the compounds of the present invention are administered as pharmaceuticals to subjects, they can be given per se or as a pharmaceutical composition containing, for example, 0.1 to 99% (more preferably, 10 to 30%) of active ingredient in combination with a pharmaceutically acceptable carrier.

Combination with a Blood Product and/or an Agent

Pharmaceutical compositions for preventing or treating the PH in the subject in need thereof are disclosed. The pharmaceutical composition comprises an effective amount of RRx-001, or a pharmaceutically acceptable salt thereof, and at least one pharmaceutically acceptable carrier or excipient. In some embodiments, the pharmaceutical composition preventing or treating PH in the subject comprises (1) an effective amount of RRx-001, or a pharmaceutically acceptable salt thereof and (2) at least one of a blood product and an additional agent.

In some embodiments, the blood product comprises erythrocyte cells. In some embodiments, the erythrocyte cells have not undergone any manipulation selected from the group consisting of genetic modification, electroporation, conjugation through biotin, conjugation to a cell-penetrating peptide, conjugation to hemoglobin, dimethyl sulfoxide osmotic pulse, endocytosis and hypotonic preswelling, hypotonic dilution, and hypo-osmotic dialysis. In some embodiments, the blood product is a mixture of packed red blood cells. In other embodiments, the blood product is whole blood. In some embodiments, the whole blood is autologous whole blood.

In some embodiments, each additional agent is an agent for treating the PH. In some embodiments, each additional agent is selected from the group consisting of: a nitrite, a PDE-5 inhibitor, a calcium channel blocker, a prostacyclin pathway agonist, an endothelin receptor antagonist, a diuretic, oxygen, a guanylate cyclase stimulator, and an anti-coagulant. In some embodiments, each additional agent is the PDE-5 inhibitor, which is selected from the group consisting of: avanafil, lodenafil, mirodenafil, sildenafil, tadalafil, vardenafil, udenafil, zaprinast, and icariin. In some embodiments, each additional agent is the prostacyclin pathway agonist, which is selected from the group consisting of: epoprostenol, treprostinil, iloprost, and selexipag. In some embodiments, each additional agent is the endothelin receptor antagonist, which is selected from the group consisting of bosentan, macitentan, ambrisentan, sibotentan, sitaxsentan and tezosentan. In some embodiments, each additional agent is a guanylate cyclase activator or riociguat.

In some embodiments, the additional agent(s) in the pharmaceutical composition are subject to a reduced incidence of drug-drug interaction as compared to direct administration of the same additional agent(s) at the same dose without being mixed with the blood product prior to administration. In certain embodiments, the reduced incidence of drug-drug interaction permits the use of a second agent that would have otherwise been contraindicated.

The present invention can provide methods of attenuating interactions of a first drug (e.g., a first therapeutic agent) and a second drug (e.g., a second therapeutic agent) in a mammal. As described herein, interactions of drugs, or drug-drug interactions, can refer to the changes of the effects of a drug or a pharmaceutical composition on a mammal when the pharmaceutical composition is taken together with a second drug or second pharmaceutical composition. In some embodiments, the interactions can occur when more than two drugs are concurrently in a mammal, regardless of the time between the administrations of the two or more drugs and thereby, and react with each other.

In some embodiments, as described herein, “attenuating interactions” of drugs refers to actions that result in reducing or preventing any types of interactions between two or more drugs or reducing the hypersensitivity, the toxicity, or adverse effects that are caused by the interactions of two or more drugs. In some embodiments, the interactions can include, but are not limited to, synergistic or antagonistic interactions, By way of examples, attenuating interactions of the drugs can be at least any one of the following scenarios: reducing and/or preventing drug-drug physical interactions, reducing and/or preventing drug-drug pharmacokinetic interactions, reducing and/or preventing the hypersensitivity caused by co-existence of the drugs, reducing and/or preventing the toxicity caused by co-existence of drugs, or reducing and/or preventing the antagonistic interactions of drugs.

In some embodiments, the effects of the attenuated interactions can be delayed, decreased, or enhanced absorption of either pharmaceutical composition, and thereby decreases or increases the action of one or more of the additional agent(s) or the pharmaceutical composition. In some embodiments, the attenuated interactions can impact the transport or the distribution of the additional agent(s) or the pharmaceutical compositions.

Accordingly, in certain embodiments, the subject has reduced incidence and/or severity of side effects compared to subjects receiving a direct administration of the same additional agent(s) at the same dose without being mixed with the blood product prior to administration. In certain embodiments, the subject has reduced side effects compared to subjects receiving a direct administration of the same additional agent(s) at the same dose without being mixed with the blood product prior to administration. In certain embodiments, the dose of the additional agent(s) in the pharmaceutical composition is at least about 10% to about 300% more than the dose recommended for a direct administration of the same additional agent(s) without being mixed with the blood product prior to administration. In certain embodiments, the dose of the additional agent(s) in the pharmaceutical composition is at least 1%, at least 5%, at least 10%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, at least 1000%, or higher, inclusive of all ranges and subranges therebetween, more than the dose recommended for a direct administration of the same additional agent(s) without being mixed with the blood product prior to administration.

In certain embodiments, the additional agent(s) has/have a longer circulating half-life in the subject compared to direct administration of the same additional agent(s) at the same dose without being mixed with the blood product prior to administration. In certain embodiments, the circulating half-life of the additional agent(s) is at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 110%, 110%, 120%, 130%, 140%, 150%, 160%, 170%, 180%, 190%, 200%, 250%, 300%, 350%, 400%, 450%, 500%, 600%, 700%, 800%, 900%, 1000%, or more, longer than the circulating half-life of the same additional agent(s) at the same dose without being mixed with the blood product before administration.

### EXAMPLES

In order that this disclosure may be more fully understood, the following Examples are set forth. It should be understood that these Examples are for illustrative purposes only and are not to be construed as limiting this disclosure in any manner.

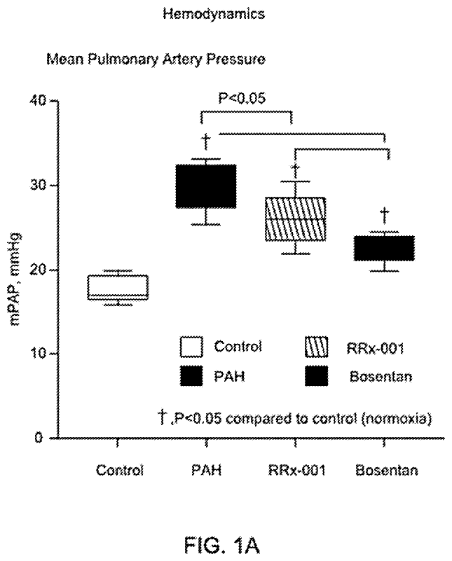

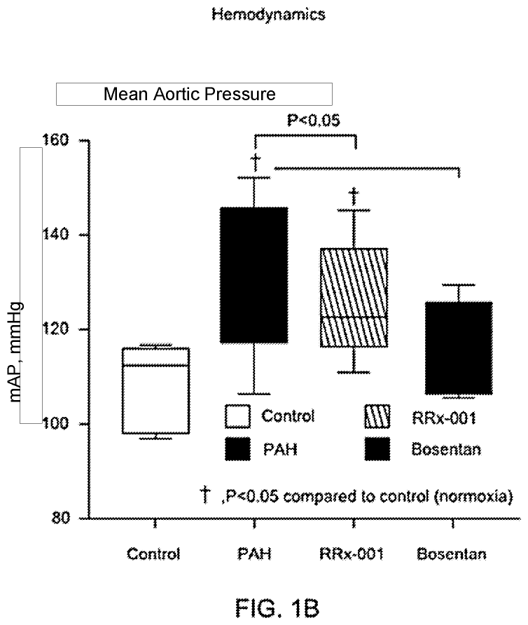

Referring to FIG. 1, FIG. 1 shows two graphs: a first graph, FIG. 1A, which is a distribution of collected data of mean pulmonary artery pressure (mPAP) of male Wistar rats from various groups of a study; and FIG. 1B, showing mean aortic pressure (mAP) of male Wistar rats from various groups of the same study. The study exposed 18 male Wistar rats to an isobaric chamber with intermittent hypoxia where the fraction of inspired oxygen (FiO₂) is 10%.

The male Wistar rats exposed to hypoxia were then divided into 3 groups for the study. A first group of 6 male Wistar rats were untreated and labeled “PAH”. A second group of 6 male Wistar rats were treated with the RRx-001. A third group of 6 male Wistar rats were treated with Bosentan, which is a drug that is used to treat PAH. The results of the three groups were compared to a control that were not exposed to the hyperbaric chamber (normoxia).

Treatment for the second group of male Wistar rats comprised mixing 5 mg/kg RRx-001 with blood and administering the mixture via tail injection before the hypoxia exposure. Similarly, treatment for the third group of male Wistar rats comprised mixing 50 mg/kg Bosentan with blood and administering the mixture daily via tail injection before the hypoxia exposure.

The mPAP was recorded in FIG. 1A using a catheter introduced into the right jugular vein of the male Wistar rats and passed across the tricuspid valve and a right ventricle into the pulmonary artery. A distribution of the mPAP results for the various groups is plotted in FIG. 1A.

The PAH group distribution has an upper value for the mPAP of approximately 33 mmHg, a median value for the mPAP of approximately 30 mmHg, and a lower value for the mPAP of approximately 27 mmHg. The RRx-001 group has an upper value for the mPAP of approximately 28 mmHg, a median value for the mPAP of approximately 26 mmHg, and a lower value for the mPAP of approximately 23 mmHg. The p value as determined for the results between the PAH group and the RRx-001 group is less than 0.05.

The Bosentan group distribution has an upper value for the mPAP of approximately 24 mmHg, a median value for the mPAP of approximately 22 mmHg, and a lower value for the mPAP of approximately 21 mmHg. The control group distribution has an upper value for the mPAP of approximately 20 mmHg, a median value for the mPAP of approximately 17 mmHg, and a lower value for the mPAP of approximately 16 mmHg.

The three trial groups exposed to the hypoxia, the PAH, the RRx-001, and Bosentan, have a p value of less than 0.05 as compared to the control. The results show that the mPAP of the RRx-001 group was between the PAH and the Bosentan. All three trial groups have a higher mPAP value than the control group.

The second graph, FIG. 1B, is a distribution of mean aortic pressure (mAP) that was recorded by an arterial catheter inserted via the carotid artery of the male Wistar rats. The PAH group distribution has an upper value for mAP of approximately 147 mmHg, a median value for the mAP of approximately 137 mmHg, and a lower value for the mAP of approximately 117 mmHg. The RRx-001 group has an upper value for the mAP of approximately 134 mmHg, a median value for the mAP of approximately 121 mmHg, and a lower value for the mAP of approximately 116 mmHg. The p value as determined for the mAP results between the PAH group and the RRx-001 group is less than 0.05.

The Bosentan group distribution has an upper value for the mAP of approximately 126 mmHg, a median value for the mAP of approximately 120 mmHg, and a lower value for the mAP of approximately 108 mmHg. The control group distribution has an upper value for the mAP of approximately 116 mmHg, a median value for the mAP of approximately 113 mmHg, and a lower value for the mAP of approximately 98 mmHg.

For the PAH and the RRx-001 groups, the p value as compared to the control is less than 0.05. The results show that the mAP of the RRx-001 group was between the PAH and the Bosentan. All three trial groups exposed to the hypoxia have a higher median mAP value than the control group.

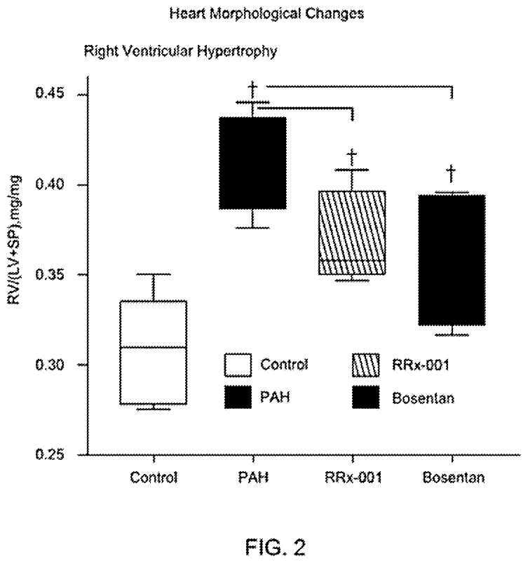

Referring to FIG. 2, FIG. 2 shows a graph of collected data of a wet weight ratio, or Fulton index, of a right ventricle (RV) against a left ventricle (LV) plus septum (SP), RV/(LV+SP). A distribution of the collected data is plotted on the graph for the 4 groups of male Wistar rats: the control group, the PAH group, the RRx-001 group, and the Bosentan group. The calculated wet weight ratio is intended to be an indicator of right ventricular hypertrophy.

To measure the wet weight ratio, the heart was excised, the atria were removed, and the wall of the right ventrical (RV) was separated from the heart. Tissues were blotted and weighed. The pulmonary arterial blood pressures increased significantly after 2 weeks of the hypoxia. The elevated pulmonary arterial pressure was paralleled by an increase in systolic pressure, and right ventricular hypertrophy according to the Fulton index.

The PAH group distribution has an upper value for the wet weight ratio of approximately 0.44, a median value for the wet weight ratio of approximately 0.43, and a lower value for the wet weight ratio of approximately 0.38. The RRx-001 group has an upper value for the wet weight ratio of approximately 0.39, a median value for the wet weight ratio of approximately 0.35, and a lower value for the wet weight ratio of approximately 0.34.

The Bosentan group distribution has an upper value for the wet weight ratio of approximately 0.39, a median value for the wet weight ratio of approximately 0.35, and a lower value for the wet weight ratio of approximately 0.32. The control group distribution has an upper value for the wet weight ratio of approximately 0.33, a median value for the wet weight ratio of approximately 0.31, and a lower value for the wet weight ratio of approximately 0.28.

All three trial groups that were exposed to the hypoxia have a p value of less than 0.05 as compared to the control. The results show that the median wet weigh ratio of the RRx-001 group was less than PAH and substantially the same as the Bosentan median value. All three trial groups exposed to the hypoxia have a higher median wet weight ratio than the control group indicating that all three groups have varying degrees of right ventricular hypertrophy.

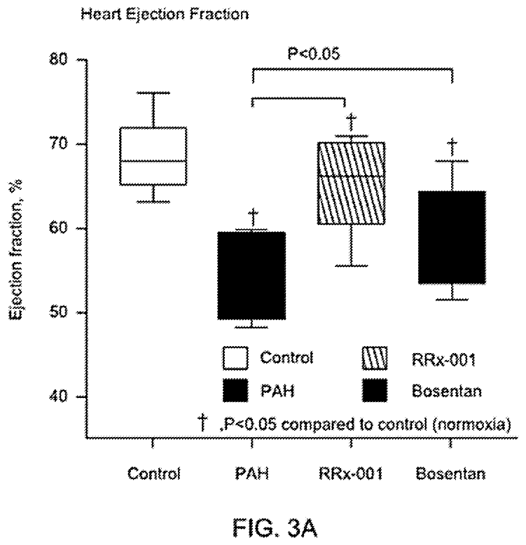

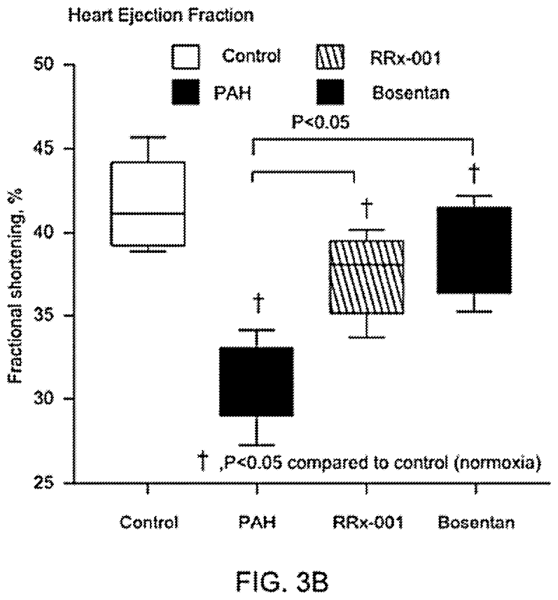

Referring to FIG. 3, FIG. 3 shows two graphs: a first graph, FIG. 3A, which is a distribution of collected data of an ejection fraction based on measurements from an echocardiograph and FIG. 3B, which is a distribution of collected data of fractional shortening based on measurements from the echocardiograph. The PAH group distribution has an upper value ejection fraction of approximately 59%, a median value ejection fraction of approximately 51%, and a lower value ejection fraction of approximately 49%. The RRx-001 group has an upper value ejection fraction of approximately 69%, a median value ejection fraction of approximately 66%, and a lower value ejection fraction of approximately 60%.

The Bosentan group distribution has an upper value ejection fraction of approximately 63%, a median value ejection fraction of approximately 59%, and a lower value ejection fraction of approximately 52%. The control group distribution has an upper value ejection fraction of approximately 72%, a median value ejection fraction of approximately 68%, and a lower value ejection fraction of approximately 65%.

All groups have a p value of less than 0.05 when compared to the control. The RRx-001 data has a p value of less than 0.05 when compared to the PAH group. As shown in FIG. 3A, the RRx-001 group has the highest heart ejection fraction of the 3 trial groups that were exposed to hypoxia and only marginally less than the control group heart ejection fraction.

In FIG. 3B, the PAH group distribution has an upper value fractional shortening of approximately 33%, a median value fractional shortening of approximately 31%, and a lower value fractional shortening of approximately 28%. The RRx-001 group has an upper value fractional shortening of approximately 39%, a median value fractional shortening of approximately 38%, and a lower value fractional shortening of approximately 35%.

The Bosentan group distribution in FIG. 3B has an upper value fractional shortening of approximately 42%, a median value fractional shortening of approximately 38%, and a lower value fractional shortening of approximately 36%. The control group distribution has an upper value fractional shortening of approximately 44%, a median value fractional shortening of approximately 42%, and a lower value fractional shortening of approximately 39%.

All groups in FIG. 3B have a p value of less than 0.05 when compared to the control. The RRx-001 data has a p value of less than 0.05 when compared to the PAH group. As shown in FIG. 3B, the RRx-001 group has median fractional shortening value that is substantially similar to the Bosentan group.

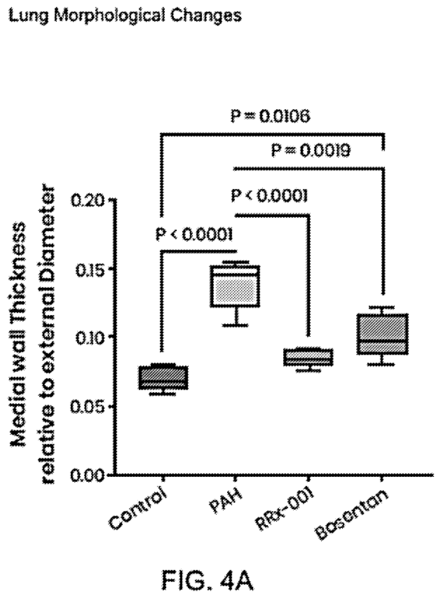

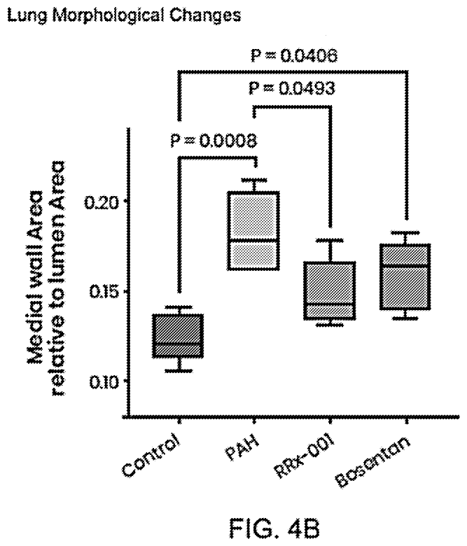

Referring to FIG. 4, FIG. 4 shows two graphs quantifying lung morphological changes in sample groups: a first graph, FIG. 4A, which is a distribution of collected data of medial wall thickness of removed lungs from male Wistar rats relative to external diameter and a second graph, FIG. 4B, which is a distribution of collected data of medial wall area relative to lumen area of removed lungs from male Wistar rats. To collect the data, male Wistar rats lungs were removed and fixed in paraformaldehyde, then dehydrated and embedded in paraffin for sectioning. The relative medial thickness and relative medial areas were calculated from vessels showing a clearly defined external and internal elastic lamina.

The PAH group distribution in FIG. 4A has an upper value medial wall thickness relative to external diameter of approximately 0.15, a median value of relative medial wall thickness of approximately 0.14, and a lower value of relative medial wall thickness of approximately 0.13. The RRx-001 group has an upper value of relative medial wall thickness of approximately 0.09, a median value of relative medial wall thickness of approximately 0.08, and a lower value of relative medial wall thickness of approximately 0.07.

The Bosentan group distribution has an upper value relative medial wall thickness of approximately 0.12, a median value relative medial wall thickness of approximately 0.10, and a lower value relative medial wall thickness of approximately 0.09. The control group distribution has an upper value relative medial wall thickness of approximately 0.08, a median value relative medial wall thickness of approximately 0.07, and a lower value relative medial wall thickness of approximately 0.07.

The RRx-001 group in FIG. 4A, has the lowest median value for relative medial wall thickness of the 3 trial groups and is only marginally higher than the median value for the control.

The PAH group distribution in the FIG. 4B, has an upper value medial wall area relative to lumen area of approximately 0.21, a median value of relative medial wall area of approximately 0.17, and a lower value of relative medial wall area of approximately 0.16. The RRx-001 group has an upper value for relative medial wall area of approximately 0.16, a median value for relative medial wall area of approximately 0.14, and a lower value for relative medial wall area of approximately 0.14.

The Bosentan group distribution has an upper value for relative medial wall area of approximately 0.17, a median value for relative medial wall area of approximately 0.16, and a lower value for relative medial wall area of approximately 0.14. The control group distribution has an upper value for relative medial wall area of approximately 0.14, a median value for relative medial wall area of approximately 0.12, and a lower value for relative medial wall area of approximately 0.11.

The RRx-001 group in FIG. 4B has the lowest median value for medial wall area relative to lumen area of the 3 trial groups that were exposed to hypoxia. Thus, male Wistar rats that were treated with the RRx-001 had the lowest lung morphological changes relative to treatment with Bosentan and the PAH group.

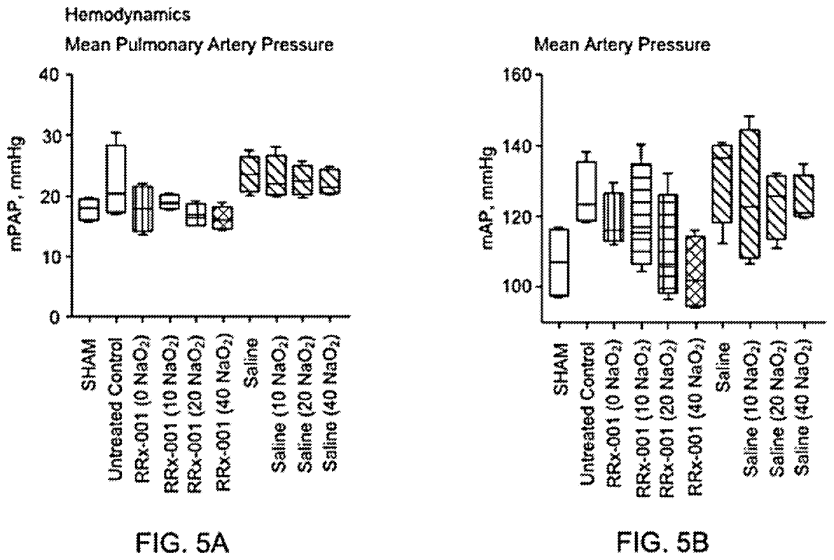

Referring to FIG. 5A, FIG. 5A is a graph of a distribution of collected data of mPAP of male Wistar rats from various groups of a study that included administration of nitrite. Male Wistar rats were kept in an isobaric hypoxic chamber with intermittent hypoxia (FiO2=10%) for (1) week (6 hours/day, 6 day/week). Forty (40) male Wistar rats (180-220 g), which were subjected to Hypoxia, were treated with the RRx-001 (10 mg/kg) and different concentrations of nitrite (0, 10, 20, and 40 mg/kg) (n=4 per group). For administration, the RRx-001 (10 mg/kg) was mixed with blood and injected via the tail at the beginning of hypoxia exposure every day. Nitrite, in the form of NaNO₂, was administered subcutaneously before administration of the RRx-001. Untreated control animals were not administered any agents and the SHAM (Control) group was not subjected to hypoxia.

Mean pulmonary pressure (mPAP) was recorded using a catheter introduced in the right jugular vein and passed across the tricuspid valve and right ventricle into the pulmonary artery.

FIG. 5A shows that median values of mPAP tended to decrease for groups to which more nitrite was administered. The groups that were administered the RRx-001 and 0, 10 mg/kg, 20 mg/kg, and 40 mg/kg nitrite have a median mPAP of approximately 17 mmHg, 18 mmHg, 16 mmHg, and 15 mmHg respectively. The groups that were administered saline and 0, 10 mg/kg, 20 mg/kg, and 40 mg/kg nitrite have a median mPAP of approximately 23 mmHg, 22 mmHg, 22 mmHg, and 20 mmHg respectively. The control has a median mPAP of approximately 20 mmHg and the SHAM group has a mPAP of approximately 17 mmHg. Accordingly, it was observed that administration of the RRx-001 and nitrite tends to result in lower mPAP measurements. Further, the largest dose of nitrite of 40 mg/kg is correlated with the greatest reduction in mPAP.

Referring to FIG. SB, FIG. 5B is a graph of a distribution of collected data of mAP of male Wistar rats from various groups of a study that included administration of nitrite. Mean aortic pressure (mAP) was recorded by an arterial catheter inserted via the carotid artery.

The groups that were administered the RRx-001 and 0, 10 mg/kg, 20 mg/kg, and 40 mg/kg nitrite have a median mAP of approximately 116 mmHg, 115 mmHg, 107 mmHg, and 102 mmHg respectively. The groups that were administered saline and 0, 10 mg/kg, 20 mg/kg, and 40 mg/kg nitrite have a median mAP of approximately 137 mmHg, 122 mmHg, 126 mmHg, and 120 mmHg respectively. The control has a median mAP of approximately 124 mmHg and the SHAM group has a median mPAP of approximately 109 mmHg. Like the data collected in FIG. 5A, it was observed that administration of the RRx-001 and nitrite tends to result lower mAP measurements.

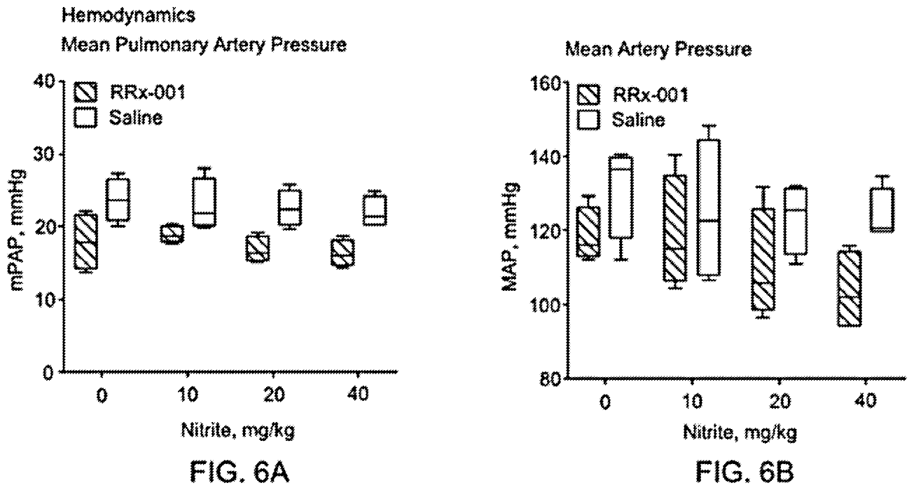

Referring to FIG. 6A and FIG. 6B, FIG. 6A is a graph of a distribution of collected data of mPAP based on nitrite dose of male Wistar rats from various groups of a study that included administration of nitrite. FIG. 6A is based on the same data shown in FIG. 5A, but plotted based on nitrite dose. FIG. 6B is a graph of a distribution of collected data of mAP based on nitrite dose of male Wistar rats from various groups of a study that included administration of nitrite. FIG. 6B is based on the same data shown in FIG. 5B, but plotted based on nitrite dose. Based on the side-by-side comparisons in FIG. 6A and FIG. 6B, nitrite seems to have a systemic effect on mAP but less significant on mPAP.

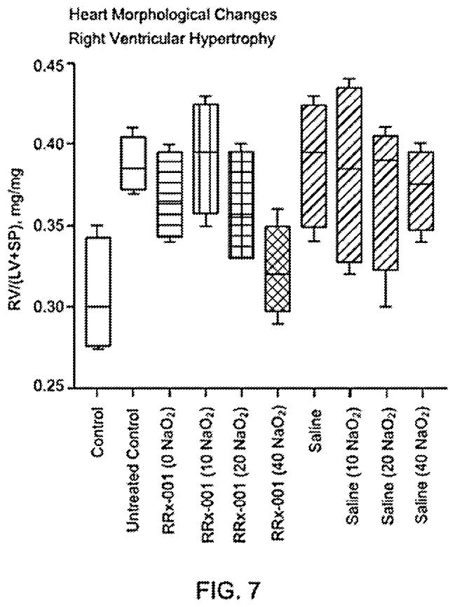

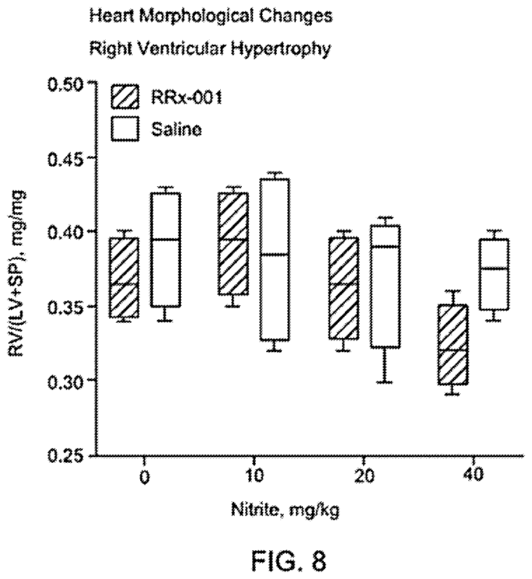

Referring to FIG. 7 and FIG. 8, FIG. 7 shows collected data of a wet weight ratio of RV/(LV+SP) from various groups of a study that included administration of nitrite. The wet weight ratio is an indicator of right ventricle hypertrophy. The wet weight ratios were measured according to the same procedure as set out in the description for FIG. 2. FIG. 8 shows a graph of the same collected data as in FIG. 7, but with the RRx-001 and saline groups plotted side-by-side for easier comparison.

The groups that were administered the RRx-001 with 0, 10 mg/kg, 20 mg/kg, and 40 mg/kg nitrite have a median wet weight ratio of approximately 0.37, 0.40, 0.35, and 0.32 mg/mg respectively. The groups that were administered the saline and 0, 10 mg/kg, 20 mg/kg, and 40 mg/kg nitrite have a median wet weight ratio of approximately 0.40, 0.38, 0.39, and 0.37 mg/mg respectively. The untreated control has a wet weight ratio of approximately 0.38 mg/mg and the control has a wet weight ratio of approximately 0.30 mg/mg.

The data show that the RRx-001 has an effect on right ventricle hypertrophy. The greatest reduction in wet weight ratio, and closest wet weight ratio to the control, is observed for the group that was administered the RRx-001 and 40 mg/kg nitrite.

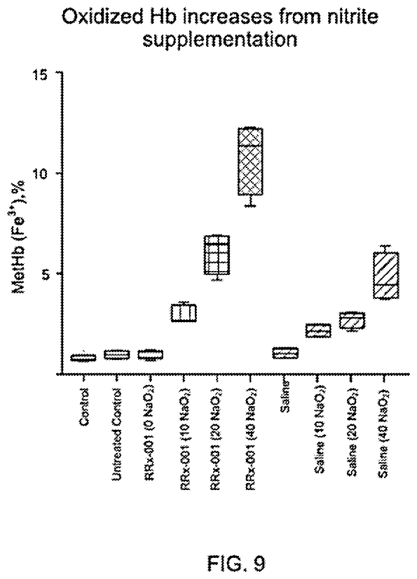

Referring to FIG. 9, FIG. 9 shows a graph of a distribution of collected data of methemoglobin (metHb) in the blood of Wistar rats. Treatment with the RRx-001 and nitrite increases metHb, which may not be good for the patient because iron (Fe) in the heme group is in the +3 oxidation state rather than the +2 oxidation state of normal hemoglobin. However, metHb may be treated with methylene blue.

The groups of Wistar rats that were administered the RRx-001 and 0, 10 mg/kg, 20 mg/kg, and 40 mg/kg nitrite have a median metHb concentration of approximately 1%, 3%, 7%, and 12% respectively. The groups that were administered saline and 0, 10 mg/kg, 20 mg/kg, and 40 mg/kg nitrite have a median metHb concentration of approximately 1%, 2%, 3%, and 5% respectively. Both the control and untreated control groups have a median metHb concentration of approximately 1%. Accordingly, administration of nitrite by itself is correlated to an increase in metHb. Also, administration of the RRx-001 with nitrite is correlated to an increase in metHb that is substantially greater than nitrite by itself.

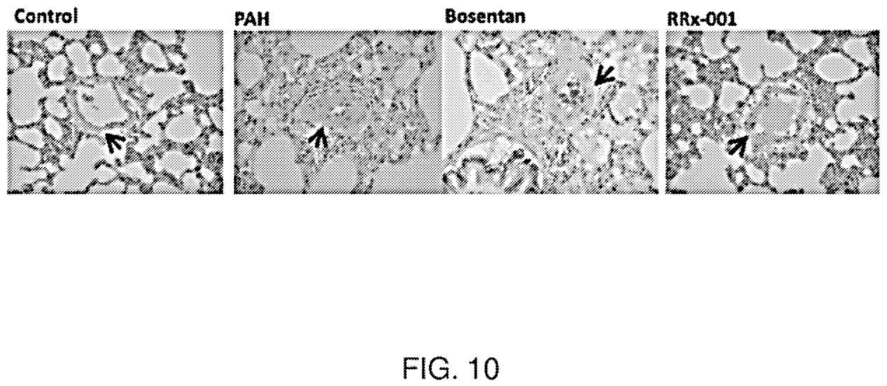

FIG. 10 depicts the effect of the RRx-001 and Bosentan on pulmonary vascular remodeling. Specifically, FIG. 10 depicts images showing H&E staining in the pulmonary arteriole. These results demonstrate that the increases in the pulmonary vascular wall thickness and muscularization of the pulmonary arteriole were inhibited by the administration of the RRx-001. These results suggest that the RRx-001 alleviates pulmonary remodeling in hypoxia induced PAH in subjects (e.g., rats).

Many variations may be made to the embodiments described herein. All variations, including combinations of embodiments, are intended to be included within the scope of this disclosure. The description of the embodiments herein can be practiced in many ways. Any terminology used herein should not be construed as restricting the features or aspects of the disclosed subject matter. The scope should instead be construed in accordance with the appended claims.

What is claimed is:

1. A method for preventing or treating pulmonary hypertension (PH) in a subject, the method comprising administering to the subject in need thereof an effective amount of RRx-001, or a pharmaceutically acceptable salt thereof.

2. The method of claim 1, wherein the effective amount of RRx-001, or the pharmaceutically acceptable salt thereof, is a therapeutically effective amount of the RRx-001, or the pharmaceutically acceptable salt thereof.

3. The method of claim 2, wherein the therapeutically effective amount of the RRx-001, or the pharmaceutically acceptable salt thereof, is in a range of about 0.1 mg and about 500.0 mg.

4. The method of claim 3, wherein the therapeutically effective amount of the RRx-001, or the pharmaceutically acceptable salt thereof, is in a range of about 0.5 mg and about 200.0 mg.

5. The method of claim 3, wherein the therapeutically effective amount of the RRx-001, or the pharmaceutically acceptable salt thereof, is in a range of about 5 mg and about 50 mg.

6. The method of claim 3, wherein the therapeutically effective amount of the RRx-001, or the pharmaceutically acceptable salt thereof, is in a range of about 10 mg and about 30 mg.

7. The method of claim 1, wherein the subject is a mammal subject.

8. The method of claim 7, wherein the mammal subject is a human subject.

9. The method of claim 7, wherein the mammal subject is a non-human subject.

10. The method of claim 1, wherein the PH is of World Health Organization (WHO) Class I, II, III, IV or V.

11. The method of claim 1, wherein the PH is associated with a condition selected from the group consisting of: congenital left to right intracardiac shunts, portal hypertension, persistent pulmonary hypertension of the newborn, collagen vascular diseases, HIV infection, exposure to drugs, and exposure to toxins.

12. The method of claim 1, wherein the PH is selected from the group consisting of: secondary to left heart disease, valvular heart disease, and restrictive cardiomyopathy.

13. The method of claim 1, wherein PH is associated with mitral valve stenosis or left heart diastolic dysfunction.

14. The method of claim 1, wherein the PH is secondary to advanced chronic lung disease or environmental hypoxia.

15. The method of claim 14, wherein the advanced chronic lung disease or environmental hypoxia comprises chronic obstructive pulmonary disease (COPD), interstitial lung disease, sleep-disordered breathing, or alveolar hypoventilation disorder.

16. The method of claim 1, wherein the PH is secondary to at least one of chronic thrombotic disease and embolic disease.

17. The method of claim 1, wherein the PH is secondary to a metabolic disorder, and wherein the metabolic disorder is selected from the group consisting of: glycogen storage disease, thyroid disease, Gaucher disease, systemic diseases such as sarcoidosis, vasculitis, neurofibromatosis type 1, hematologic diseases such as myeloproliferative disorders as well as end-stage renal disease on dialysis, extrinsic compression of pulmonary vessels, and embolization of tumors.

18. The method of any one of claims 1-17, wherein administering the effective amount of RRx-001, or the pharmaceutically acceptable salt thereof, occurs via oral administration, transdermal administration, administration by inhalation, nasal administration, topical administration, intra-aural administration, rectal administration, intravenous administration, intramuscular administration, subcutaneous administration, or intraperitoneal administration, or combinations thereof.

19. The method of any one of claims 1-17, wherein administering the effective amount of RRx-001, or the pharmaceutically acceptable salt thereof, occurs via parenteral administration.

20. The method of any one of claims 2-19, wherein the therapeutically effective amount of the RRx-001, or the pharmaceutically acceptable salt thereof, results in an amelioration of at least one adverse event of the PH in the subject.

21. The method of claim 20, wherein each adverse event of the at least one adverse event is selected from the group consisting of: dyspnea, exercise intolerance, systemic venous congestion, ascites and lower extremity edema.

22. The method of claim 20, wherein each adverse event of the at least one adverse event is selected from the group consisting of: cardiac remodeling and pulmonary remodeling.

23. The method of claim 20, wherein each adverse event of the at least one adverse event comprises a decreased ejection fraction.

24. The method of any one of claims 2-19, wherein the therapeutically effective amount of the RRx-001, or the pharmaceutically acceptable salt thereof, results in a reduction in a probability of death.

25. The method of any one of claims 2-19, wherein administering the therapeutically effective amount of RRx-001, or the pharmaceutically acceptable salt thereof, results in peripheral vasodilation in the subject.

26. The method of any one of claims 2-19, wherein administering the therapeutically effective amount of RRx-001, or the pharmaceutically acceptable salt thereof, results in an increased exercise tolerance as compared to a baseline.

27. The method of any one of claims 2-19, wherein administering the therapeutically effective amount of RRx-001, or the pharmaceutically acceptable salt thereof, results in a decreased dyspnea as compared to a baseline.

28. The method of any one of claims 2-19, wherein administering the therapeutically effective amount of RRx-001, or the pharmaceutically acceptable salt thereof, results in a reduction of vascular thickening in the subject as compared to a baseline.

29. The method of any one of claims 2-19, wherein administering the therapeutically effective amount of RRx-001, or the pharmaceutically acceptable salt thereof, results a decrease in inflammatory cell infiltration into a lumen in the subject as compared to a baseline.

30. The method of any one of claims 2-19, wherein the therapeutically effective amount of the RRx-001, or the pharmaceutically acceptable salt thereof, results in a reduction in a probability of hospitalization.

31. The method of any one of claims 2-19, wherein administering the therapeutically effective amount of RRx-001, or the pharmaceutically acceptable salt thereof, results in antioxidation in the subject.

32. The method of any one of claims 1-31, wherein administering the therapeutically effective amount of RRx-001, or the pharmaceutically acceptable salt thereof, is performed via a single administration.

33. The method of any one of claims 1-31, wherein administering the therapeutically effective amount of RRx-001, or the pharmaceutically acceptable salt thereof, is performed via at least two administrations.

34. The method of claim 33, wherein administering the therapeutically effective amount of RRx-001, or the pharmaceutically acceptable salt thereof, is performed at a frequency of between about 1 time per hour and about 1 time per month.

35. The method of claim 33, wherein administering the therapeutically effective amount of RRx-001, or the pharmaceutically acceptable salt thereof, is performed at a frequency of between about 4 times per day and about 1 time per week.

36. The method of claim 33, wherein administering the therapeutically effective amount of RRx-001, or the pharmaceutically acceptable salt thereof, is performed at a frequency of between about 2 times per day and about 3 times per week.

37. The method of claim 33, wherein administering the therapeutically effective amount of RRx-001, or the pharmaceutically acceptable salt thereof, is performed at a frequency of about one time a day.

38. The method of any of claims 1-37, further comprising:
administering an agent before, during, or after the administration of the effective amount of RRx-001, or the pharmaceutically acceptable salt thereof.

39. The method of claim 38, wherein the agent comprises an agent for treating PH.

40. The method of claim 38, wherein the agent is selected from the group consisting of: a nitrite, a PDE-5 inhibitor, a calcium channel blocker, a prostacyclin pathway agonist, an endothelin receptor antagonist, a diuretic, oxygen, a guanylate cyclase stimulator, and an anti-coagulant.

41. The method of claim 40, wherein the agent comprises the PDE-5 inhibitor, and wherein the PDE-5 inhibitor is selected from the group consisting of avanafil, lodenafil, mirodenafil, sildenafil, tadalafil, vardenafil, udenafil, zaprinast, and icariin.

42. The method of claim 40, wherein the agent comprises the prostacyclin pathway agonist, and wherein the prostacyclin pathway agonist is selected from the group consisting of epoprostenol, treprostinil, iloprost, and selexipag.

43. The method of claim 40, wherein the agent comprises the endothelin receptor antagonist, and wherein the endothelin receptor antagonist is selected from the group consisting of bosentan, macitentan, ambrisentan, sibotentan, sitaxsentan and tezosentan.

44. The method of claim 38, wherein the agent comprises a guanylate cyclase activator or riociguat.

45. The method of any of claims 1-44, wherein the therapeutically effective amount of the RRx-001, or the pharmaceutically acceptable salt thereof, is administered as a composition comprising a blood product.

46. The method of claim 45, wherein the blood product comprises erythrocyte cells.

47. The method of claim 46, wherein the erythrocyte cells have not undergone any manipulation selected from the group consisting of genetic modification, electroporation, conjugation through biotin, conjugation to a cell-penetrating peptide, conjugation to hemoglobin, dimethyl sulfoxide osmotic pulse, endocytosis and hypotonic preswelling, hypotonic dilution, and hypo-osmotic dialysis.

48. The method of claim 45, wherein the blood product is a mixture of packed red blood cells.

49. The method of claim 45, wherein the blood product is whole blood.

50. The method of claim 45, wherein the whole blood is autologous whole blood or donor-matched allogenic whole blood.

51. A composition for preventing or treating pulmonary hypertension (PH) in a subject in need thereof, the composition comprising an effective amount of RRx-001, or a pharmaceutically acceptable salt thereof, and at least one pharmaceutically acceptable carrier or excipient.

52. The composition of claim 51, wherein the effective amount of RRx-001, or the pharmaceutically acceptable salt thereof, is a therapeutically effective amount of the RRx-001, or the pharmaceutically acceptable salt thereof.

53. The composition of claim 52, wherein the therapeutically effective amount of the RRx-001, or the pharmaceutically acceptable salt thereof, is in a range of about 0.1 mg and about 500.0 mg.

54. The composition of claim 53, wherein the therapeutically effective amount of the RRx-001, or the pharmaceutically acceptable salt thereof, is in a range of about 0.5 mg and about 200.0 mg.

55. The composition of claim 53, wherein the therapeutically effective amount of the RRx-001, or the pharmaceutically acceptable salt thereof, is in a range of about 5 mg and about 50 mg.

56. The composition of claim 53, wherein the therapeutically effective amount of the RRx-001, or the pharmaceutically acceptable salt thereof, is in a range of about 10 mg and about 30 mg.

57. The composition of claim 51, wherein the subject is a mammal subject.

58. The composition of claim 57, wherein the mammal subject is a human subject.

59. The composition of claim 57, wherein the mammal subject is a non-human subject.

60. The composition of claim 51, wherein the PH is of World Health Organization (WHO) Class I, II, III, IV or V.

61. The composition of claim 51, wherein the PH is associated with a condition selected from the group consisting of: congenital left to right intracardiac shunts, portal hypertension, persistent pulmonary hypertension of the newborn, collagen vascular diseases, HIV infection, exposure to drugs, and exposure to toxins.

62. The composition of claim 51, wherein the PH is selected from the group consisting of: secondary to left heart disease, valvular heart disease, and restrictive cardiomyopathy.

63. The composition of claim 51, wherein PH is associated with mitral valve stenosis or left heart diastolic dysfunction.

64. The composition of claim 51, wherein the PH is secondary to advanced chronic lung disease or environmental hypoxia.

65. The composition of claim 64, wherein the advanced chronic lung disease or environmental hypoxia comprises chronic obstructive pulmonary disease (COPD), interstitial lung disease, sleep-disordered breathing, or alveolar hypoventilation disorder.

66. The composition of claim 51, wherein the PH is secondary to at least one of chronic thrombotic disease and embolic disease.

67. The composition of claim 51, wherein the PH is secondary to a metabolic disorder, and wherein the metabolic disorder is selected from the group consisting of: glycogen storage disease, thyroid disease, Gaucher disease, systemic diseases such as sarcoidosis, vasculitis, neurofibromatosis type 1, hematologic diseases such as myeloproliferative disorders as well as end-stage renal disease on dialysis, extrinsic compression of pulmonary vessels, and embolization of tumors.

68. The composition of any one of claims 51-67, wherein the effective amount of RRx-001, or the pharmaceutically acceptable salt thereof, is administered via oral administration, transdermal administration, administration by inhalation, nasal administration, topical administration, intra-aural administration, rectal administration, intravenous administration, intramuscular administration, subcutaneous administration, or intraperitoneal administration, or combinations thereof.

69. The composition of any one of claims 51-67, wherein the effective amount of RRx-001, or the pharmaceutically acceptable salt thereof, is administered via parenteral administration.

70. The composition of any one of claims 52-69, wherein administration of the therapeutically effective amount of the RRx-001, or the pharmaceutically acceptable salt thereof, results in an amelioration of at least one adverse event of the PH in the subject.

71. The composition of claim 70, wherein each adverse event of the at least one adverse event is selected from the group consisting of: dyspnea, exercise intolerance, systemic venous congestion, ascites and lower extremity edema.

72. The composition of claim 70, wherein each adverse event of the at least one adverse event is selected from the group consisting of: cardiac remodeling and pulmonary remodeling.

73. The composition of claim 70, wherein each adverse event of the at least one adverse event comprises a decreased ejection fraction.

74. The composition of any one of claims 52-73, wherein administration of the therapeutically effective amount of the RRx-001, or the pharmaceutically acceptable salt thereof, results in a reduction in a probability of death.

75. The composition of any one of claims 52-74, wherein administration of the therapeutically effective amount of RRx-001, or the pharmaceutically acceptable salt thereof, results in peripheral vasodilation in the subject.

76. The composition of any one of claims 52-75, wherein administration of the therapeutically effective amount of RRx-001, or the pharmaceutically acceptable salt thereof, results in an increased exercise tolerance as compared to a baseline.

77. The composition of any one of claims 52-76, wherein administration of the therapeutically effective amount of RRx-001, or the pharmaceutically acceptable salt thereof, results in a decreased dyspnea as compared to a baseline.

78. The composition of any one of claims 52-77, wherein administration of the therapeutically effective amount of RRx-001, or the pharmaceutically acceptable salt thereof, results in a reduction of vascular thickening in the subject as compared to a baseline.

79. The composition of any one of claims 52-78, wherein administration of the therapeutically effective amount of RRx-001, or the pharmaceutically acceptable salt thereof, results a decrease in inflammatory cell infiltration into a lumen in the subject as compared to a baseline.

80. The composition of any one of claims 52-79, wherein administration of the therapeutically effective amount of the RRx-001, or the pharmaceutically acceptable salt thereof, results in a reduction in a probability of hospitalization.

81. The composition of any one of claims 52-80, wherein administration of the therapeutically effective amount of RRx-001, or the pharmaceutically acceptable salt thereof, results in antioxidation in the subject.

82. The composition of any one of claims 52-81, wherein the therapeutically effective amount of RRx-001, or the pharmaceutically acceptable salt thereof, is administered via a single administration.

83. The composition of any one of claims 52-81, wherein the therapeutically effective amount of RRx-001, or the pharmaceutically acceptable salt thereof, is administered via at least two administrations.

84. The composition of claim 83, wherein the therapeutically effective amount of RRx-001, or the pharmaceutically acceptable salt thereof, is administered at a frequency of between about 1 time per hour and about 1 time per month.

85. The composition of claim 83, wherein the therapeutically effective amount of RRx-001, or the pharmaceutically acceptable salt thereof, is administered at a frequency of between about 4 times per day and about 1 time per week.

86. The composition of claim 83, wherein the therapeutically effective amount of RRx-001, or the pharmaceutically acceptable salt thereof, is administered at a frequency of between about 2 times per day and about 3 times per week.

87. The composition of claim 83, wherein the therapeutically effective amount of RRx-001, or the pharmaceutically acceptable salt thereof, is administered at a frequency of about one time a day.

88. The composition of any one of claims 51-87, further comprising an agent.

89. The composition of claim 88, wherein the agent comprises an agent for treating PH.

90. The composition of claim 89, wherein the agent is selected from the group consisting of: a nitrite, a PDE-5 inhibitor, a calcium channel blocker, a prostacyclin pathway agonist, an endothelin receptor antagonist, a diuretic, oxygen, a guanylate cyclase stimulator, and an anti-coagulant.

91. The composition of claim 90, wherein the agent comprises the PDE-5 inhibitor, and wherein the PDE-5 inhibitor is selected from the group consisting of avanafil, lodenafil, mirodenafil, sildenafil, tadalafil, vardenafil, udenafil, zaprinast, and icariin.

92. The composition of claim 90, wherein the agent comprises the prostacyclin pathway agonist, and wherein the prostacyclin pathway agonist is selected from the group consisting of epoprostenol, treprostinil, iloprost, and selexipag.

93. The composition of claim 90, wherein the agent comprises the endothelin receptor antagonist, and wherein the endothelin receptor antagonist is selected from the group consisting of bosentan, macitentan, ambrisentan, sibotentan, sitaxsentan and tezosentan.

94. The composition of claim 88, wherein the agent comprises a guanylate cyclase activator or riociguat.

95. The composition of any one of claims 51-94, further comprising a blood product.

96. The composition of claim 95, wherein the blood product comprises erythrocyte cells.

97. The composition of claim 96, wherein the erythrocyte cells have not undergone any manipulation selected from the group consisting of genetic modification, electroporation, conjugation through biotin, conjugation to a cell-penetrating peptide, conjugation to hemoglobin, dimethyl sulfoxide osmotic pulse, endocytosis and hypotonic preswelling, hypotonic dilution, and hypo-osmotic dialysis.

98. The composition of claim 95, wherein the blood product is a mixture of packed red blood cells.

99. The composition of claim 95, wherein the blood product is whole blood.

100. The composition of claim 99, wherein the whole blood is autologous whole blood or donor-matched allogenic whole blood.
